(19) United States
(12) Patent Application Publication
(10) Pub. No.: US 2024/0342066 A1
(43) Pub. Date: Oct. 17, 2024

(54) HIGH POTENCY EMULSIONS

(71) Applicant: Vertosa Inc., Walnut Creek, CA (US)

(72) Inventor: CHUNXIAO HAN, El Cerrito, CA (US)

(21) Appl. No.: 18/750,843

(22) Filed: Jun. 21, 2024

Related U.S. Application Data
(63) Continuation of application No. 18/553,969, filed on Jan. 1, 0001.
(60) Provisional application No. 63/173,965, filed on Apr. 12, 2021.

(51) Int. Cl. A61K 8/34; A23D 7/005; A23G 3/40; A23G 3/48; A23G 3/54; A23L 2/52; A61K 8/06; A61K 8/9789; A61Q 19/00

(52) U.S. Cl. CPC A61K 8/347; A23D 7/0053; A23G 3/40; A23G 3/48; A23G 3/54; A23L 2/52; A61K 8/06; A61K 8/9789; A61Q 19/00; A61K 2800/10

(57) ABSTRACT

Provided are high-potency emulsions with one or more active agents that can be infused into a product to produce a product with the one or more active agents.

### CLAIM OF PRIORITY UNDER 35 U.S.C. § 119

The present application is a continuation application of U.S. application Ser. No. 18/553,969 filed on Nov. 4, 2023, which is a U.S. National Stage entry under U.S.C. 371 of International Application No. PCT/US2022/071661, filed on Apr. 11, 2022, designating the United States of America and published in English on Oct. 20, 2022, which in turn claims benefit of U.S. Provisional Application No. 63/173,965, entitled “HIGH POTENCY EMULSIONS” filed Apr. 12, 2021. The entire contents of each of the foregoing is hereby incorporated by reference herein.

### BACKGROUND

### Field

The invention relates to a high-potency emulsion with an active agent that can be infused into a product to produce a product with an active agent.

### Background

Aqueous-based Cannabis products are becoming increasingly popular. Such products avoid the unhealthy effects of smoking and can deliver cannabinoids efficiently and reliably in a discrete manner.

### SUMMARY

Some embodiments of the invention relate to a product infused with an emulsion composition. The emulsion composition can include droplets that can include one or more active agents, a carrier oil, an emulsifier, and water. The emulsion composition can have a high potency, for example at least 30 mg/g or 40 mg/g. The emulsifier can be a Quillaja extract. The emulsion is stable in the product.

In some embodiments, the droplet size increases by less than 10% after heating the emulsion composition for 48 hours at 55° C.

In some embodiments, the carrier oil can be at least 0.3 times the one or more active agents by weight. In some embodiments, the emulsifier can be at least 0.05 times the total amount of the one or more active agent and carrier oil by weight. In some embodiments, the water can be at least the total amount of the one or more active agents and the emulsifier.

In some embodiments, the carrier oil is derived from plants or animals.

In some embodiments, the carrier oil can be selected from sunflower oil, olive oil, coconut oil, sesame oil, avocado oil, palm oil, soybean oil, corn oil, peanut oil, canola oil, grape seed oil, corn oil, hazelnut oil, rice bran oil, linseed oil, safflower oil, sesame oil, passion fruit oil, lard, butter, cheese, animal fat, and/or the like, and/or combinations thereof.

In some embodiments, the one or more active agents is a cannabinoid or a terpene.

In some embodiments, the cannabinoid can be selected from the group consisting of tetrahydrocannabinolic acid A (THCA-A), tetrahydrocannabinolic acid B (THCA-B), tetrahydrocannabinol (THC), tetrahydrocannabinolic acid C (THCA-C), tetrahydrocannbinol C (THC-C), tetrahydrocannabi varinic acid (THCVA), tetrahydrocannabivarin (THCV), tetrahydrocannabiorcolic acid (THCA-C), tetrahydrocannabiorcol (THC-C), delta-7-cis-iso-tetrahydrocannabivarin, A-tetrahydrocannabinolic acid (A8-THCA), A-tetrahydrocannabinol (A-THC), cannabidiolic acid (CBDA), cannabidiol (CBD), cannabidiol monomethyl ether (CBDM), cannabidiol-C (CBD-C), cannabidivarinic acid (CBDVA), cannabidivarin (CBDV), cannabidiorcol (CBD-C), cannabigerolic Acid (CBGA), cannabigerolic Acid monomethylether (CBGAM), cannabigerol (CBG), cannabigerol monomethylether (CBGM), cannabigerovarinic Acid (CBGVA), cannabigerovarin (CBGV), cannabichromenic Acid (CBCA), cannabichromene (CBC), cannabichromevarinic Acid (CB CVA), cannabichromevarin (CBCV), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C (CBN-C), cannabivarin (CBV), cannabino-C (CBN-C), cannabiorcol (CBN-C), cannabinodiol (CBND), cannabinodivarin (CBDV), cannabitriol (CBT), 10-Ethoxy-9-hydroxy-A″-tetrahydrocannabinol, 8.9-dihydroxy-A′-tetrahydrocannabinol (8.9-Di-OH CBT-C), cannabitriolvarin (CBTV), ethoxy-cannabitriolvarin (CBTVE), dehydrocannabifuran (DCBF), cannbifuran (CBF), cannabichromanon (CBCN), cannabicitran (CBT), 10-Oxo-A′-tetrahydrocannabinol (OTHC), A-cis-tetrahydrocannabinol (cis-THC), cannabiripsol (CBR), 3,4,5,6-tetrahydro-7-hydroxy-alpha-alpha-2-trim ethyl-9-n-propyl-2,6-methano-2H-1-benzoxocin-5-methanol (OH-iso-HHCV), Trihydroxy-delta-9-tetrahydrocannabinol (triCH-THC), isocannabinoids, and combinations thereof.

In some embodiments, the product is an oral spray, a candy, a small volume beverage shot, or a powder. In some embodiments, the product is a cosmetic product.

In some embodiments, the product can be organically certified according to current USDA guidelines.

In some embodiments, the product can have an onset time of less than 30 minutes.

In some embodiments, the product can be a beverage of less than 15 mL with a potency of at least 1 mg/mL.

Some embodiments of the invention relate to a method for producing the product disclosed herein. The method can include one or more of: providing an emulsion composition comprising one or more active agents, a carrier oil, Quillaja extract, and water; and homogenizing the emulsion composition with a base beverage.

In some embodiments, the product is a candy including a gummy shell and a liquid center, where the candy has potency of at least 1 mg/g.

Some embodiments of the invention relate to a method for producing a candy including a gummy shell and a liquid center. The method can include one or more of: providing an emulsion composition comprising one or more active agents, a carrier oil, Quillaja extract, and water; and homogenizing the emulsion composition with a base composition for a gummy shell.

In some embodiments, the product is a powder capable of consumption.

Some embodiments of the invention relate to a method for producing a powder capable of consumption. In some embodiments, the method can include providing an emulsion composition comprising one or more active agents, a carrier oil, Quillaja extract, and water; providing a plating agent; and homogenizing the emulsion composition with the plating agent.

### BRIEF DESCRIPTION OF THE DRAWINGS

FIG. 1 shows results from a pharmacokinetic study of different emulsions.

FIG. 2 depicts results from a dosage delivery study comparing different emulsions.

FIG. 3 depicts results from a compatibility study of different emulsions in a cosmetic product.

FIG. 4. depicts results from a compatibility study of different emulsions in a mouthwash.

FIG. 5 depicts results from a freeze-thaw stability study of different emulsions.

### DETAILED DESCRIPTION

As disclosed herein are methods and compositions for creating products infused with a high potency emulsion. The invention relates to a high potency emulsion that can be infused into products with a high target potency but have low total volume. Examples of these products include low volume beverages, candies, aerosol products, sprayed products, cosmetics, any product described herein, and the like.

As used herein, an infused product is a product that has been incorporated with another product. The emulsion infused product described herein is a product where the emulsion has been incorporated into the product. The incorporation can be done by mixing a base product with the emulsion in a liquid solution or by spraying the emulsion onto a base product or any other similar method.

The emulsion technology can introduce one or more hydrophobic active agents (or “actives”) into an aqueous base product. An active agent or an “active” can be defined as a molecule or a set of molecules capable of modifying or modulating a biological system. The product can have a fast onset and increased bioavailability compared to a tincture that directly uses cannabinoid in carrier oil. For example, the product can have an onset time of about 10 to 30 minutes (e.g., 10, 12, 14, 16, 18, 20, 22, 24, 26, 28, or 30 minutes). For example, the product can have a bioavailability of about 10% up to 95% (e.g., 10, 15, 20, 25, 30, 35, 40, 45, 50, 55, 60, 65, 70, 75, 80, 85, 90, or 95%), depending on food consumption (i.e., depending on how much food is in a consumer's stomach).

The emulsion can include one or more of a hydrophobic active agent, a carrier oil, a main emulsifier, an optional co-emulsifier, water, and/or other optional additional ingredients such as a stabilizer or a preservative.

TABLE 1

| | | | Optional | | Optional | |
|---|---|---|---|---|---|---|
| Hydrophobic | Carrier | Main | Co- | | Preservative | Total |
| Active Agent | Oil | Emulsifier | Emulsifier | Water | or Stabilizer | Weight |
| A | B | C | D | E | F | G = |
| | | | | | | (A + B + C + |
| | | | | | | D + E + F) |

The term “emulsion,” as used herein, can refer to a mixture of two or more liquids that are not usually miscible or soluble with one another.

The term “active agent” as used herein, can refer to a substance that can produce a chemical reaction.

The term “emulsifier” as used herein, can refer to a substance that can stabilize the emulsion.

The hydrophobic active agent can be a cannabinoid, a terpene, an essential oil, a flavonoid, a polyphenol, and any combination thereof.

Exemplary cannabinoids can include, but are not limited to tetrahydrocannabinolic acid A (THCA-A), tetrahydrocannabinolic acid B (THCAB), tetrahydrocannabinol (THC), tetrahydrocannabinolic acid C (THCA-C), tetrahydrocannabinol C (THC-C), tetrahydrocannabivarinic acid (THCVA), tetrahydrocannabivarin (THCV), tetrahydrocannabiorcolic acid (THCA-C), tetrahydrocannabiorcol (THC-C), delta-7-cis-iso-tetrahydrocannabivarin, delta-8-tetrahydrocannabinolic acid (Δ8-THCA), delta-9-tetrahydrocannabinol (Δ9-THC), cannabidiolic Acid (CBDA), cannabidiol (CBD), cannabidiol monomethylether (CBDM), cannabidiol-C (CBD-C), cannabidivarinic acid (CBDVA), cannabidivarin (CBDV), cannabidiorcol (CBD-C), cannabigerolic acid (CBGA), cannabigerolic acid monomethylether (CBGAM), cannabigerol (CBG), cannabigerol monomethylether (CBGM), cannabigerovarinic Acid (CBGVA), cannabigerovarin (CBGV), cannabichromenic Acid (CBCA), cannabichromene (CBC), cannabichromevarinic Acid (CBCVA), cannabichromevarin (CBCV), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabinolic acid (CBNA), cannabinol (CBN), cannabinol methylether (CBNM), cannabinol-C4 (CBN-C4), cannabivarin (CBV), cannabinol-C (CBN-C), cannabiorcol (CBN-C1), cannabinodiol (CBND), cannabinodivarin (CBVD), cannabitriol (CBT), 10-Ethoxy-9-hydroxy-delta-6a-tetrahydrocannabinol, 8,9-dihydroxy-delta-6a-tetrahydrocannabinol (8,9-Di-OH-CBT-C5), cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (Δ9-cis-THC), cannabiripsol (CBR), -3,4,5,6-tetrahydro-7-hydroxy-alpha-alpha-2-trimethyl-9-n-propyl-2,6-methano-2H-1-benzoxocin-5-methanol (OH-iso-HHCV), trihydroxy-delta-9-tetrahydrocannabinol (triOH-THC), an isocanabinoid, any other cannabinoid, and any combination thereof.

Where this application describes a cannabinoid, other active agents can be used instead of or in addition to the cannabinoid.

Exemplary terpenes can include, but are not limited to, myrcene, limonene, linalool, beta-caryophyllene, alpha-pinene and beta-pinene, alpha-bisabolol, cucalyptol, trans-nerolidol, humulene, delta-3-carene, camphene, borneol, terpineol, valencene, geraniol, eugenol, sabinene, phellandrene, borneol, isoborneol, phytol, menthol, geraniol, citronellol, ocimene, halomon, thymol, carvacrol, thujene, camphene, camphor, verbenone, botrydial, ngaione, cuparane, labdane, ferruginol, cafestol, any other terpene, and any combination thereof.

Exemplary essential oils can include but are not limited to vitamin E; vitamin B12; vitamin A; vitamin D; vitamin B; omega 3; astaxanthin; fish oil; medium chain triglyceride (MCT) oil; long chain triglyceride (LCT) oil; cannabinoid(s) in MCT; coconut oil; palm oil; eicosapentaenoic acid (EPA); docosahexaenoic acid (DHA); essential oils such as but not limited to lemon oil, orange oil, peppermint oil, Ylang-Ylang oil, lemongrass oil, tea tree oil, rosemary oil, Australian sandalwood oil, grapefruit oil, frankincense oil, cedarwood oil, patchouli oil, cinnamon bark oil, bergamot oil, chamomile oil, lemon-eucalyptus oil, ginger oil, key lime oil, vanilla oil, clove oil; any other essential oil; and any combination thereof.

Exemplary flavonoids can include, but are not limited to cannflavin A, cannflavin B, cannflavin C, orientin, quercetin, silymarin, kaempferol, apigenin, any other flavonoid, and any combination thereof.

Exemplary polyphenols can include, but are not limited to cannabism B, caffcoyltyramine, canniprene, any other polyphenol, and any combination thereof.

Exemplary carrier oils can include, but are not limited to, sunflower oil, olive oil, coconut oil, sesame oil, avocado oil, palm oil, soybean oil, corn oil, peanut oil, canola oil, grape seed oil, corn oil, hazelnut oil, rice bran oil, linseed oil, safflower oil, sesame oil, passion fruit oil, lard, butter, cheese, animal fat, medium chain triglyceride (MCT) oil, long chain triglyceride (LCT) oil, cannabinoid(s) in MCT, vitamin E, vitamin B12, vitamin A, vitamin D, vitamin B, omega 3, astaxanthin, fish oil, eicosapentaenoic acid (EPA), docosahexaenoic acid (DHA), any other carrier oil, and any combination thereof.

In some embodiments, essential oils are used in combination with the carrier oil. The role of the essential oil is to reduce the viscosity of the oil phase to match it with the viscosity of the water phase. As the viscosities of the two phases get closer, it can be easier to break the interfacial surface and decrease droplet size.

In some embodiments, the emulsion includes a main emulsifier and a co-emulsifier. The main emulsifier can be defined as the emulsifier that is present in a larger concentration. The main emulsifier can include an extract of Quillaja (also referred to as Quillaja extract or Quillaia extract). The co-emulsifier can include another Quillaja extract, polysorbate, polyglycerol (10-2-P), gum acacia, Q-Naturale®, vitamin E TPGS, lecithin, sucrose ester, any other emulsifier, and any combination thereof.

Some embodiments of the invention relate to a “Quillaja extract Cannabis emulsion” or a “Quillaja extract based Cannabis emulsion” or a “Quillaja based Cannabis emulsion” wherein the emulsion includes Quillaja extract as the main emulsifier and an active agent found in Cannabis.

Commercially available Quillaja extract, such as E 999, is obtained by aqueous extraction of the milled inner bark or wood of Quillaja saponaria, other Quillaja species, or trees of the family Rosaceae. It contains a number of triterpenoid saponins consisting of glycosides of quillaic acid.

Quillaja extract or Quillaja extract is a natural ingredient with potential to be used in products that can be organically certified. Other names can include Murillo bark extract, Panama bark extract, Quillay bark extract, and Soapbark extract.

Ingredion® is another supplier of a Quillaja extract with the commercial name of Q-Naturale®, which is a 20% Quillaja extract water solution. There are 4 major types of Q-Naturale® that offer different features such as preservative, organic certification, vegan certification, and natural sediment. Table 2 summarizes these types.

TABLE 2
Q-Naturale ® types and their features

| | Code | Preservative | Organic | Vegan | Sediment |
|---|---|---|---|---|---|
| Q-Naturale ® | 100 | Yes | No | No | Less |
| | 200 | No | No | No | Less |
| | 200V | No | No | Yes | Heavy |
| | 300 | No | Yes | No | Less |

Quillaja extract can also be delivered in other commercial products such as SAPNOV™ series from Naturex® and Q Ultra® or QDP Ultra® series from Desert King™. Quillaja extract can be obtained in a dry powder form or an aqueous form. The dilution factors for either form factor depends on the active content of the Quillaja extract.

The optional additional ingredients of the emulsion can include preservatives, antioxidants, electrolytes, stabilizers, pH modulators, flavor agents, coloring agents, and/or the like.

Exemplary preservatives or stabilizers can include but are not limited to ethyl lauroyl arginate, sodium bi-sulphite, potassium benzoate, potassium sorbate, ascorbic acid, citric acid, benzoic acid, sodium benzoate, calcium ascorbate, erythorbic acid, sodium ascorbate, sorbic acid, sulphurous acid, calcium sorbate, vitamin C, vitamin E, any other preservative, and any combination thereof.

In some embodiments, the antioxidant can be a vitamin. Vitamins can include, but are not limited to, vitamin A (retinol), vitamin C (ascorbic acid), and vitamin E (tocopherol). In some embodiments, the antioxidant can be a carotenoid terpenoid such as, but not limited to, alpha or beta carotene, astaxanthin, cryptoxanthin, lutein, lycopene, zeaxanthin, or canthaxanthin; phenolic acids and their esters, such as, but not limited to, chicoric acid, chlorogenic acid, cinnamic acid, ellagic acid, ellagitannins, gallic acid, salicylic acid, rosmarinic acid, and gallotannins; nonflavonoid phenolics such as, but not limited to, curcumin, flavonolignans, xanthones, or eugenol; and/or flavonoids such as, but not limited to flavones, flavonols, flavanones, stilbenoids, isoflavone phytoestrogens, and anthocyanins. Other non-limiting examples of antioxidants can include capsaicin, bilirubin, citric acid, oxalic acid and phytic acid, EDTA, TBHQ, BHA, BHT, propyl gallate, and/or the like. In some embodiments, the antioxidant can be any commercially available antioxidant such as, for example, brewshield, structuan, rosemary extract, and/or the like (e.g., Herbalox® (41.19.32) provided by Kalsec®).

Exemplary pH modulators can include but are not limited to citric acid, ascorbic acid, fumaric acid, lactic acid, phosphoric acid, acetic acid, malic acid, tartaric acid and/or any combinations thereof.

Exemplary food colors can include but are not limited to blue, green, red, purple, orange, and/or the like.

Exemplary flavoring agents can include but are not limited to honey, agave, caramel, an essential oil, a bitter blocker (e.g., ((3-[1-[(3,5-dimethylisoxazol-4-yl)methyl]pyrazol-4-yl]-1-[(3-hydroxyphenyl)methyl]imidazolidine-2,4-dione), GG-605-390-4, NP-844-232-9, QJ-6 15-696-6, TruClear®, stevia, and/or the like), a terpene, an artificial flavor agent (e.g., mint, orange, strawberry, cherry, and/or the like), and/or the like. Such ingredients can improve the taste and appearance of the composition.

The emulsion has a starting potency and is infused into a product to produce a product with a target potency.

The starting potency can be presented by active agent weight over total weight (A/G). The starting potency determines a dilution factor for the product that can be produced.

The starting potency of the emulsion can be about 20 mg/g, 30 mg/g, 40 mg/g, 50 mg/g, 60 mg/g, 70 mg/g, 80 mg/g, 90 mg/g, 100 mg/g, 110 mg/g, 120 mg/g, 130 mg/g, 140 mg/g, 150 mg/g, 160 mg/g, 170 mg/g, 180 mg/g, 190 mg/g, 200 mg/g, 210 mg/g, 220 mg/g, 230 mg/g, 240 mg/g, 250 mg/g, 260 mg/g, 270 mg/g, 280 mg/g, 290 mg/g, 300 mg/g, 310 mg/g, 320 mg/g, 330 mg/g, 340 mg/g, 350 mg/g, 360 mg/g, 370 mg/g, 380 mg/g, 390 mg/g, 400 mg/g or more. High-potency can be defined as 20 mg/g, 30 mg/g, 40 mg/g, 50 mg/g, 60 mg/g, 70 mg/g, 80 mg/g, 90 mg/g, 100 mg/g, 110 mg/g, 120 mg/g, 130 mg/g, 140 mg/g, 150 mg/g, 160 mg/g, 170 mg/g, 180 mg/g, 190 mg/g, 200 mg/g, 210 mg/g, 220 mg/g, 230 mg/g, 240 mg/g, 250 mg/g, 260 mg/g, 270 mg/g, 280 mg/g, 290 mg/g, 300 mg/g, 310 mg/g, 320 mg/g, 330 mg/g, 340 mg/g, 350 mg/g, 360 mg/g, 370 mg/g, 380 mg/g, 390 mg/g, 400 mg/g or more.

The invention discloses high-potency emulsions that can be infused into products to produce a product with an active agent. The invention relates to the surprising discovery of Quillaja extract's ability to reach that high potency.

The product can be in the form of a pill, tablet, capsule, oblong tablet, sprinkle, aerosol, powder, cream, lotion, liquid, gel, foam, solid, any of the forms described herein and/or a combination of any of the same.

The product can be any product that can be infused with the emulsion to deliver the one or more active agent to a consumer/user when the product is consumed and/or administered. Administration can be oral, topical, and/or the like.

Quillaja extract can be obtained from the Quillaja saponaria (soak bark) tree and thus offers a natural and organic option for consumers.

When looking at the emulsion composition and when a high potency emulsion is desired, the goal is to reduce the combined weight of B+C+D+E+F.

Quillaja extract can work with very low amount of carrier oil. When B is as low as 0.25A, it is enough for Quillaja extract to form a stable emulsion.

Carrier oil (B) can enrich the oil phase volume to ensure that the emulsion droplet has balanced phases. In some embodiments, (B) is 0.25 times-25 times (e.g., 0.25, 0.3, 0.35, 0.4, 0.45, 0.5, 0.6, 0.7, 0.8, 0.9 1, 2, 5, 7, 10, 12, 15, 17, 20, 22, or 25 times) of the active agent(s) amount (A).

The main emulsifier, depending on its surface activity and size, can be between 0.05 times to 5 times (e.g., 0.05, 0.5, 1, 1.5, 2, 2.5, 3, 3.5, 4, 4.5, or 5) the total amount of active agent(s) and carrier oil, or C=(0.05-5)×(A+B).

In some embodiments, a co-emulsifier can be used. The co-emulsifier can further reduce the interfacial tension, protect the oil-water boundary, and create more stable emulsions. In some embodiments, the co-emulsifier can be 0.2 to 7 (e.g., 0.2, 0.5, 1, 1.5, 2, 2.5, 4, 4.5, 5, 5.5, 6, 6.5, 7) times the main emulsifier, or (D)=(0.2-7)×(C). In other embodiments, there is no co-emulsifier.

Preservatives or stabilizers can be added to the ratio of the liquid volume. For example, sodium benzoate and potassium sorbate can be added as 0.03-0.1% (e.g., 0.03, 0.04, 0.05, 0.06, 0.07, 0.08, 0.09, or 0.1%) of the total emulsion weight; citric acid can be added 0.1-5% (e.g., 0.1, 0.25, 0.5, 1, 2.5, 2, 2.5, 3, 3.5, 4, 4.5, or 5%) of the total emulsion weight; ascorbic acid can be added 0.01%-5% (e.g., 0.1, 0.25, 0.5, 1, 2.5, 2, 2.5, 3, 3.5, 4, 4.5, or 5%) of the total emulsion weight.

Water can be the key part of determining the final potency of the emulsion. Since in an O/W emulsion, water is the continuous phase and it is usually over 50% of the total weight, in which case, (E)≥(A+B+C+D+F).

The emulsion can have a droplet size range of about 30-500 nm. For example, droplet size can be about 30, 40, 50, 60, 70, 80, 90, 100, 150, 200, 250, 300, 350, 400, 450 or 500 nm.

The potency of the active ingredient is calculated by (A)/(G). In some embodiments, the invention relates to certain infused products, where the product's volume is limited (e.g., 0.1 mL to 60 mL) but requires mid to high amount (e.g., 5 to 100 mg) of active per piece. In these embodiments, an emulsion with high active potency is used so that the emulsion can deliver the target active amount by occupying a small volume of the product. The invention relates to the use of Quillaja extract as an emulsifier that can deliver a very high target potency without sacrificing the flavor, stability, and droplet size and distribution profile.

The emulsion can be processed by a high-pressure homogenizer, such as microfluidizer, BEEi 2000, or Dyhydromatic HP-1200 at 5,000-45,000 PSI for 1-8 passes, until a translucent emulsion is achieved.

In some embodiments, essential oils are used in combination with the carrier oil.

Embodiments of the invention relate to the production of various products infused with the emulsion described herein. In general, the emulsion can be added to a “base” product. The base product can be an existing product or an intermediate product of an existing product.

### Beverage Shot

In some embodiments, the emulsion-infused product is a small volume beverage or “beverage shot.” The beverage shot can have a volume of, for example, 5 mL to 30 mL. For example, the volume can be about 5, 10, 15, 20, 25, 30 mL, or more.

The emulsion used in this application can have the following ingredients

TABLE 3
Emulsion for infusion into a beverage shot

| Ingredients | Mass (g) |
|---|---|
| Active Agent | 1 |
| Carrier oil | 0.5-3 |
| Quillaja extract | 0.075-0.2 |
| Preservative/Stabilizer | 0-2 |
| Water | 3-10 |

The beverage shot can include the emulsion described herein to have one or more active agents. The active agent(s) can be selected based on the desired “experience” Consumption of Cannabis by a human generally results in a wide variety of psychotropic effects, but which is often referred to as a “high.” The Cannabis high varies depending on many factors, including the strain of Cannabis, the amount consumed, the method of consumption, the biochemistry of the individual consuming it and the individual's level of experience in consuming Cannabis. That said, a Cannabis high can include euphoria, anxiety, a general alteration of conscious perception, feelings of well-being, relaxation or stress reduction, increased appreciation of humor, music (especially discerning its various components/instruments) or the arts, joviality, metacognition and introspection, enhanced recollection (episodic memory), increased sensuality, increased awareness of sensation, increased libido, and creativity. Abstract or philosophical thinking, disruption of linear memory and paranoia or anxiety are also typical effects. The specific experience can be designed by blending different active agents at specific ratios. This can be done by mixing different active agents into one oil phase and processing this emulsion using a single emulsifier. Alternatively, different actives can be produced into different emulsions, where the same or different emulsifiers can be applied. The resulting emulsions with different active agents can be measured and combined to certain ratios for a targeted effect, which can be packaged into a small beverage shot and sold to enhance different real-life experiences.

For example, THC, CBD, eucalyptol, and limonene can be combined into one Cannabis shot and marketed to artists to boost their creativity during work. In another example, CBD, CBG, linalool, and myrcene can be combined into one drink to help with muscle pain recovery and sold to gym members or with at-home gym equipment brands. In another example, THC, CBD, beta-caryophyllene, and linalool can be combined into one drink to boost calming and centering, which can be used in yoga practice to help people meditate more deeply. In addition, CBD and beta-caryophyllene can be combined at a certain ratio and sold to airlines so that the passengers can have a relaxing and enjoyable long-distance flight. In another example, THC, alpha pinene, limonene, and ocimene can be combined to create a beverage shot that can be sold at movie theater to enhance a customer's mood so that they can better enjoy the movie.

Quillaja extract can be used as a “universal emulsifier” due to the high potency feature of the emulsion. A universal emulsifier can describe an emulsifier that can be used in a wide variety of products. Higher starting potency makes it possible to blend in different active agents at certain ratios, while still leaving enough room for additional ingredients, including but not limited to, flavor and coloring agents, preservatives, and stabilizers.

Some embodiments of the invention relate to a method of making a beverage shots. The method can include:
Step 1. Producing a Cannabis emulsion that has one or more actives. For example, to create a beverage shot with 1:1 THC:CBD ratio, THC and CBD extract can be weighed out separately and dissolved separately into MCT as carrier oil, then Quillaja extract and water can be added separately, and the two emulsions are processed under a high shear mixer and before going through a high-pressure homogenizer at pressure level of 10,000-40,000 PSI for 1-5 passes. The higher the pressure and the more passes, the smaller the droplet size and thus the greater homogeneity of the product. Another way to produce active emulsion is to mix THC and CBD extract together while calculating their ratio to be 1:1. And then carrier oil, Quillaja extract and water are added step wise, and emulsion follows through high shear mixing and high-pressure homogenizer.
Step 2. Diluting the raw emulsion to target potency of the beverage shot. In either case mentioned above, the dilution is created to meet target potency in the final product. For example, if starting potency for the 2 separate emulsions are both at 50 mg/mL, to produce a beverage shot at 10 mg THC+10 mg CBD in 5 mL, a 25 times dilution factor is needed.
Step 3. Optionally adding other ingredients such as flavor, preservative, pH modulator, antioxidant, and/or antifoaming agent. List ingredient weight and percentage in each unit and then calculate total weight needed for total number of units to be produced. Sec Table 4 below for a non-limiting example.

TABLE 4

| Ingredients | Weight (g) | Weight to produce 100,000 shots (g) |
|---|---|---|
| THC Emulsion | 0.2 | 20,000 |
| CBD Emulsion | 0.2 | 20,000 |
| Monk Fruit Flavor | 0.8 | 80,000 |
| Sodium Benzoate | 0.0015 | 150 |
| Citric Acid | 0.2 | 20,000 |
| Ascorbic Acid | 0.01 | 1,000 |
| Potassium Sorbate | 0.0015 | 150 |
| RO Water | 3.587 | 358,700 |
| Total Weight (g) | 5 | 500,000 |

Step 4. Homogenizing all ingredients by overhead stir or high shear mixing, then dispense product into final packaging. The product can be tested for potency and microbial content, (optional as heavy metal, residual solvent, pesticide, mycotoxin, or aflatoxin), prior to being released to the market.

### Gusher Candy

In some embodiments, the emulsion-infused product is a “gusher” candy. A gusher is a product that has a hard shell (e.g., chocolate or sugar) surrounding a liquid core. The liquid core can include ingredients, such as but not limited to sugar, alcohol, and the like. A Cannabis-infused gusher has Cannabis emulsion infused into the liquid core (e.g., a delicious chocolate and then a flavorful Cannabis emulsion with quick onset), so the consumer can have an enjoyable experience.

A gusher may only have 0.3 mL for the empty volume inside. To make the emulsion flavor appealing to a consumer, it may need 0.2 mL reserved for a flavor agent or agents. In such an embodiment, the volume for the emulsion would be only 0.1 mL. If each gusher is targeting 15 mg of cannabinoid, then the starting potency would be equal or higher than 150 mg/mL (15 mg/0.1 mL). Since the emulsion density is usually around 1 g/mL, the starting emulsion potency could be equal or higher than 150 mg/g.

The emulsion used in this application can have the following ingredients:

TABLE 5
Emulsion for infusion into a gusher candy

| Ingredients | Mass (g) |
|---|---|
| Active Agent | 1 |
| Carrier oil | 0.5-10 |
| Quillaja extract | 0.075-2 |
| Preservative/Stabilizer | 0-2 |
| Water | 3-10 |

Some embodiments of the invention relate to a method of making a “gusher” candy. A gusher candy can be defined as a candy that has a sugary shell holding up a liquid core that can be released once the sugary shell melts in the mouth. In some embodiments, the invention relates to a Cannabis-infused gusher where the Cannabis can either be incorporated into the sugary shell, for example, as a chocolate or the inner liquid. Usually, the inner liquid is placed on the outside shell as a wax or solid at low temperature, and the layered material gets wrapped up and chopped into individual candy. When returned to room temperature, the wax or solid internal material melts to become liquid. In the case of applying Cannabis emulsions, the emulsion can first get mixed with another flavoring agent first and follow the similar process to get this mixture frozen at −5° C. to 0° C. for over 5-24 hours (e.g., 5.1, 5.5, 10, 15, 20, 24, or 25 hours). After taking out this material from the freezer and letting it melt into a state where it is not too hard to reshape, it can be placed onto the sugary base. There is usually 1-3 hours (e.g., 1, 1.2, 1.4, 1.6, 1.8, 2, 2.2, 2.4, 2.6, 2.8, or 3 hours) to allow this placing to happen if the environmental temperature can be controlled below 10-15° C. (e.g., 5, 6, 7, 8, 9, 10, 11, 12, 13, 14, or 15° C.). After the cannabinoid emulsion liquid mixture is placed, wrapped with sugary base, and cut into individual pieces, the following steps can be similar to produce regular gusher.

A requirement of the Cannabis emulsion in this case is the freeze-thaw stability of the emulsion, by which the freezing temperature should not cause emulsion properties to change.

### Sprayed Products

In some embodiments, the emulsion-infused product is a product that is sprayed with the emulsion. The spraying of the emulsion onto the product can be followed by a drying process where Cannabis-infused product can be produced.

The emulsion used in this application can have the following ingredients listed in Table 6:

TABLE 6
Quillaja based Cannabis emulsion for spraying onto a food product

| Ingredients | Mass (g) |
|---|---|
| Active Agent | 1 |
| Carrier oil | 0.5-1.5 |
| Quillaja extract | 0.075-0.1 |
| Preservative/Stabilizer | 0-2 |
| Water | 3-10 |

In one embodiment, the product can be a Cannabis-infused tea leaf, coffee bean, or the like. When a beverage is prepared from the Cannabis-infused tea leaf or coffee bean, the cannabinoids can be introduced into the beverage naturally. To achieve this, cannabinoids can come with their own water-soluble form. The Quillaja extract based Cannabis emulsion can be sprayed onto such water-soluble cannabinoids to add value to the product.

Some embodiments of the invention relate to a method for producing a product sprayed with the Quillaja extract emulsion. In some embodiments, the method can include creating the Quillaja extract emulsion at a certain potency level based on the final target product potency.

The starting potency of this emulsion depends on the how much spray is allowed on the target food. For example, ground coffee beans can have a large surface area, where they can absorb more liquid emulsion. In contrast, a dried banana can have certain capacity limits for absorbing liquid emulsion mists. The final target potency of the food, the spray volume, and the starting potency of the Cannabis emulsion needs to be carefully calculated to match each other. For example, a cut banana piece can roughly absorb 0.1 mL of liquid emulsion on its surface. Therefore, if the target cannabinoid potency for each piece is 5 mg, then the starting potency of the emulsion should be ˜5 mg/0.1 mL=50 mg/mL.

Then the emulsion can be evenly sprayed onto the food base. In some embodiments, the emulsion can be sprayed using a fine misting mechanism, wherein the mechanism can include a mister.

After spraying, the food base can go through a drying process. In the drying process, the temperature and humidity can be controlled to allow for the evaporation of excess moisture. Different food drying mechanisms can be used, including but not limited to freeze drying, oven drying, vacuum drying, drying on a convert belt, and/or the like. Many different kinds of food can be sprayed to create added value, such as but not limited to tea leaves, tea bags, coffee beans, grounded coffee powder, instant coffee powder, raisins, dates, prunes (dried plums), figs, apricots, peaches, mangos, pineapples, berries, banana and any other fresh or dried fruits.

### Cosmetic Product

In some embodiments, the emulsion-infused product can be a cosmetic product. For example, the product can be toner, gel, cream, hand sanitizer, nasal sprays, night cream, lotion, shaving gel, aftershave lotion, beard softeners, men's talcum, pre-shave lotions (all types), shaving soap, pain relief gel, hair gel, hair coloring, hair shampoo, lipstick, soap, bleach, body wash, skin fresheners, douches, bath soap, foundations, eye lotions, air dyes and colors, hair lightener, and color, hair gel, hair spray, hair shampoo, hair conditioner, hair lightener, hair color, hand sanitizers, hand soap, paste masks, feminine wipes, medical wipes, surgical wipes, toothpaste, mouth wash, mouth spray, floss, pads, tampons, masks, diapers, band-aids, and the like. The composition can be non-irritating, made of organic ingredients, and/or has a higher delivery rate to the epidermis layer without delivering cannabinoids into systemic circulation.

The emulsion used in this application can have the following ingredients:

TABLE 7
Emulsion for cosmetic products

| Ingredients | Mass (g) |
|---|---|
| Active Agent | 1 |
| Carrier oil | 0.5-20 |
| Quillaja Extract | 0.075-5 |
| Preservative/Stabilizer | 0-2 |
| Water | 3-20 |

The Quillaja extract Cannabis emulsion can be infused during the last step of the cosmetic production process under cold temperatures, which is called “cold processable”.

Some embodiments of the invention relate to a method of making a cosmetic product:
Step 1. Providing an emulsion with one or more active agents.
Step 2. Providing a base product. The base product can be an aqueous-based cosmetic product such as a gel, cream, or liquid (e.g., toner).
Step 3. Infusing the base product with the emulsion. The infusing step can include agitation or mixing of the base product and emulsion. The infusing step can occur under cold temperature, for example, at about 40, 35, 30, 35° F. In other embodiments, the infusing step can occur at a higher temperature, for example, at 100, 90, 80, 70, 60, 50, or 45° F.
Step 4. Optionally adding an additional ingredient. The additional ingredient can be a dye, color, fragrance, pH modulator, and/or the like. The temperature used can depend on the cosmetic base. For example, a cosmetic base with high viscosity at low temperatures can require higher temperatures to ensure even distribution of the emulsion throughout the base.
Step 5. Optionally testing the product for compatibility. For example, testing to ensure that there is no layer separation and/or sedimentation that forms over time.

Since many personal care products already contain high amounts of surface-active substance such as surfactants, those materials may not be compatible with the emulsion, which also use surfactant as the main stabilizing mechanism. The compatibility can be tested by adding an emulsion into the cosmetic products and observing physical changes such as layer separation, “O-ring formation” sedimentation, color change, pH change, viscosity change, and the like. An “O-ring” is a description for a light-colored ring that can appear on the top of the solution when a solution is placed in a container like a test tube. Formation of an O-ring can be a sign of instability: when emulsion droplets merge and become bigger, if the emulsion oil phase density is lower than water phase, it may float to the top of the solution and form a ring that resembles an O-ring seal.

The Quillaja extract Cannabis emulsion can be applied to regular skincare products, including but not limited to, night cream, lotion, shaving gel, aftershave lotion, beard softeners, men's talcum, pre-shave lotions (all types), shaving soap, pain relief gel, hair gel, hair coloring, hair shampoo, lip stick, soap, bleach, body wash, skin fresheners, douches, bath soap, foundations, eye lotions, and the like.

Thus, some embodiments of the invention relate to night cream, lotion, shaving gel, aftershave lotion, beard softeners, men's talcum, pre-shave lotions (all types), shaving soap, pain relief gel, hair gel, hair coloring, hair shampoo, lip stick, soap, bleach, body wash, skin fresheners, douches, bath soap, foundations, eye lotions, and/or the like that include the Quillaja extract Cannabis emulsion.

Some embodiments of the invention relate to hair products with the Quillaja extract Cannabis emulsion. The emulsion can add new benefits, such as anti-inflammatory effects, to the layer of skin under the hair. The invention can be used for hair washing products such as hair shampoo and conditioner; hair dying products such as hair dyes and colors, hair lightener, and color; or products that stay on hair for a considerably longer period such as hair gel and hair spray. The absorption area for Cannabis-infused hair products is usually the skin on top of the head and beneath the hair. The more spray that can reach this area and the more contact time would help achieve the beneficial effect from the Cannabis infused hair product.

Some embodiments of the invention relate to paste masks (i.e., “mud packs”) or facial masks with the Quillaja extract Cannabis emulsion. These masks can stay on user's face for 10-20 minutes during application, and there are already aqueous materials from the masks that provide the cooling and moisturizing effects for the facial skin. Some products also have mechanisms to help open pores and allow other active ingredients to penetrate more deeply into the skin, thus creating long term benefits.

Cannabinoids can be applied into hand sanitizers and hand soap to increase the antifungal and antibacterial properties of these products. For example, CBD can be selected as the active agent in hand sanitizer. See table below for the compatibility chart of the Quillaja extract Cannabis emulsion with couple main disinfectant organic solvents. The results shows that Quillaja extract Cannabis emulsion has a wide compatibility range towards many different types of disinfectant solvents.

The Quillaja extract Cannabis emulsion can also be infused into disinfectant wet wipes for both hard surface and human skin applications. For hard surface applications, certain cannabinoids can help reduce bacterial growth and maintain cleanliness for a longer period of time. For human skin applications, depending on the designated area and user age, different cannabinoids can be applied. For example, for feminine wipes, CBD and CBG can be applied due to their antibacterial and calming effects. For surgical or medical wipes, different cannabinoids can be combined to achieve desired effects such as cleanliness, sanitation, cooling, or calming.

The emulsion can be incorporated by being sprayed onto the wipe during the very last step of production or it by being introduced into the soaking mixture earlier in production. The high compatibility provides the foundation for disinfectant applications of Quillaja extract emulsion to infuse cannabinoids.

TABLE 8

| Organic Solvents | Quillaja extract based |
|---|---|
| with Disinfectant Properties | Cannabis Emulsion |
| Methanol | Compatible up to 1:1 |
| Ethanol | Compatible up to 1:1 |
| Isopropanol | Compatible up to 1:1 |
| Hydrogen Peroxide | Compatible up to 1:1 |

The invention also includes other personal care products (e.g., deodorant, foot cream, sunscreen, shaving gel, face scrub, moisturizer) that benefit from high potency in small volumes, easy infusion, and wide ingredient compatibility of the Quillaja extract Cannabis emulsion. The infusion mechanism can be very similar to infusion mechanisms of mainstream cosmetic products. The Quillaja extract Cannabis emulsion can be infused at the very last step of a manufacturing process after the compatibility is evaluated before the production. Adding cannabinoids into personal care products may dramatically improve their quality and even create a new product category due to their improved efficacy and functionality. For example, the antibacterial properties of cannabinoids would work well with personal care products like deodorant and foot cream. In addition, the anti-inflammatory properties would offer new and marketable features to sunscreen, shaving gel, face scrub, and moisturizer.

The Quillaja extract Cannabis emulsions can also be introduced into products that related to the health of the eyes, nose, and mouth.

The emulsion can be infused into eye drops or eye cream. Eye drops are usually very limited in their volume, thus the Quillaja extract Cannabis emulsion can be an advantage. Potential benefits of adding a cannabinoid emulsion to eye drops can include relaxation and calming effects.

Some embodiments of the invention relate to methods for making an eye drop infused with a Quillaja extract Cannabis emulsion. Usually, eye drops contain limited amount of liquid and in some cases, 50-90% of the volume is water. To infuse an active agent such as a cannabinoid into an eye drop, the starting emulsion needs to have very high potency. For example, one eye drop could have 5 mL as total volume with 80% of water and 20% actives not including cannabinoid. If each application takes 0.2 mL, and the target potency is 5 mg of total cannabinoids, the overall potency of the eye drop should be 5 mg/0.2 mL=25 mg/mL. For a total of 5 mL eye drop, there should be 5 mL×25 mg/mL=125 mg total cannabinoids. Since emulsion contains water as part of the ingredient, it can be utilized to dissolve other active ingredients. In this case, 5 mL×80%=4 mL and the starting emulsion should have a potency of 125 mg/4 mL=31.25 mg/mL.

In some embodiments, for any of the products described herein, if more than one active agent is used, the method can include adding each active agent separately so that each ingredient dissolves well before adding a new ingredient.

Some embodiments relate to nasal sprays or sinus rinses including a Quillaja extract Cannabis emulsion. Nasal sprays work by delivering the active medicine through the mucus system within the nasal area.

Sinus rinses function to clean up the nasal cavity, usually with the help of saline. Both nasal sprays and sinus rinses operate under an aqueous condition and can benefit from infusion with an active agent such as a cannabinoid. For example, the antibacterial effect of certain cannabinoids can help clean the nasal cavity, which could also reduce the infection of airborne viruses. The anti-inflammatory property of the cannabinoid emulsion would increase the value of the nasal spray.

Some embodiments of the invention relate to making a sinus rinse including the Quillaja extract Cannabis emulsion. In some embodiments, the sinus rinse is added to a hardware device adapted for a sinus rinse. Both a liquid Quillaja extract Cannabis emulsion or an Quillaja extract Cannabis emulsion derived dry powder can be used. The dry powder can be obtained by freeze-drying, spray-drying, or apply the Quillaja extract Cannabis emulsion onto a porous subtract. In other embodiments, powdered material is used and dissolved it directly into a bottle. In this approach, the dry powder is first mixed well with another key powdery ingredient such as sodium chloride. A powder mixer can be capable of delivering the homogenous result within 0.5-5 hours of mixing. The powder can be tested for potency homogeneity, where multiple samples are taken from different parts of the product and ensure cannabinoids potency is within the passing range (<5% variation).

The Cannabis emulsion can also benefit oral cavity health products, such as toothpaste, mouth wash, and floss. The Quillaja extract Cannabis emulsion can be added into the toothpaste during any step that introduces water. The emulsion can replace the water infusion step. A compatibility study can be performed to ensure that the original toothpaste ingredients are compatible with the Quillaja extract Cannabis emulsion. The high potency feature of the emulsion helps reduce the amount of water that would otherwise be introduced. Since the emulsion potency can vary based on the internal Quillaja extract and carrier oil ratio, the desired starting potency can be customized for particular product requirements.

Some embodiments of the invention relate to making a toothpaste with a Quillaja extract Cannabis emulsion. In some embodiments, the method includes limiting water content in the emulsion since water can reduce the viscosity of the product. This can be achieved with the Quillaja extract Cannabis emulsion to support a high oil load with low water amount. In some embodiments, water content is reduced by using low to high heat (35-75° C.) to cause water to evaporate from product.

The emulsion can also be infused into mouthwash or mouth spray to, for example, increase the antibacterial and anti-inflammatory properties. The liquid in the spray can be a simple Cannabis distillate and/or isolate with carrier oil such as MCT. This product will have a slower onset time and lower total bioavailability. The liquid can also be aqueous based, where Cannabis emulsion can be applied. When sprayed in mouth, the small droplet size can facilitate the absorption of cannabinoid(s) in the buccal and sublingual tissues in the mouth thus enter the systemic circulation. This offers a quicker onset and higher bioavailability.

Since mouth spray is designed to be portable, it requires the total volume to be small. The total liquid volume can be 20 mL, or 15 mL, or 10 mL or 5 mL or 2.5 mL. The volume of each spray can be 0.5 mL, or 0.4 mL, or 0.3 mL or 0.2 mL or 0.1 mL or 0.05 mL. Each spray can deliver a certain amount of cannabinoid(s). The flavor component can take up to 20-50% of the total volume, leaving 50-80% of the rest volume for cannabinoid(s). For a micro-dose mouth spray, each spray can deliver 3 mg, or 2 mg, or 1 mg, or 0.5 mg total cannabinoid(s). For a low dose mouth spray, each spray can deliver 4 mg, or 5 mg, or 6 mg, or 7 mg, or 8 mg, or 9 mg total cannabinoid(s). For a high dose mouth spray, each spray can deliver 10 mg-20 mg total cannabinoid(s).

Some methods of the invention relate to producing a mouthwash or spray with a Quillaja based Cannabis emulsion. The method can include mixing the Cannabis emulsion with a base product. The method can include adding flavoring agents and stirring. The method can include adding additional ingredients such as melatonin, lions' mane, caffeine, taurine, boswellia, alcohol, L-theanine, ashwagandha and other functional terpenes such as myrcene, limonene, linalool, beta-caryophyllene, alpha-pinene and beta-pinene, alpha-bisabolol, cucalyptol, trans-nerolidol, humulene, delta-3-carene, camphene, borneol, terpineol, valencene, geraniol, eugenol, sabinene, phellandrene, borneol, isoborneol, phytol, menthol, geraniol, citronellol, ocimene, halomon, thymol, carvacrol, thujene, camphene, camphor, verbenone, botrydial, ngaione, cuparane, labdane, ferruginol, cafestol, any other terpene, and any combination thereof. If certain functional or flavor ingredients are not water soluble, it may need to dissolve in a carrier such as ethanol or propylene glycol or be delivered in a different or similar emulsion system. After all ingredients are mixed, stirring, or high shear mixing can be used to ensure homogeneity of the batch. In some embodiments, citric acid or preservatives such as sodium benzoate or potassium sorbate can be applied. The product can then be dispensed into each mouth spray container. The product can be tested for potency, microbial and other required parameters and then release to market.

In some embodiments for producing a mouthwash, the emulsion is added after all the other active ingredients of the mouthwash are added. After emulsion is added, the ingredients can be mixed by stirring or high shear mixing for 0.5-5 hours to ensure all ingredients are mixed well together. Then it can be ready to be dispensed into mouthwash packaging. If ethanol is included in the mouthwash, make sure not to add it together with emulsion, in which case may have detrimental effect to emulsion stability. Shelf life is another point of concern, when mouthwash contains enough amount of ethanol (>20%), microbial may not be an issue. For alcohol free mouthwash, low pH (<4.5) and preservatives (sodium benzoate and potassium sorbate) may be needed to ensure the product does not spoil over shelf life, especially when it is open and being applied by consumer over a long period of time.

The Quillaja extract Cannabis emulsion can also be applied to floss. The floss material can be soaked in the concentrated emulsion prior to a drying process. The drying process removes excess water while the cannabinoid(s) remain active on the floss material. The emulsion can also be sprayed onto the floss material prior to going through a drying process.

Similarly, to integrate the emulsion with floss, the highly concentrated liquid Quillaja extract Cannabis emulsion can also be applied to other personal care products where dry application is needed. For example, feminine pads, tampons, masks, band-aids, diapers, and others. Even though different applications can have slightly different infusion processes, the general logic of infusion is similar: start with a highly potent Quillaja extract Cannabis emulsion, soak the material into the emulsion or spray the emulsion evenly onto the material, and then remove excess water from the material through a drying process. For those applications, the higher the starting potency, the easier for the downstream processing.

Another alternative route is to produce an already soaked and dried linen or fiber material and integrate that material into products like pads, tampons, masks, diapers, and band-aids. Quillaja extract Cannabis emulsion can still be the ideal path to attach the cannabinoid(s) onto this special material through a spraying or soaking process followed by a drying process.

### Powder

In some embodiments, the emulsion-infused product can be a powder. The powder can be added into another powdery ingredient to formulate a final product. For example, an instant, dry, powdery beverage mix would contain the Cannabis powder, sugar, electrolytes, and other nutritional ingredients.

In this embodiment, a plating agent is used. The plating agent can be any plating agent such as N-Zorbit® 2144. The plating mechanism involves the physical binding of the active agent onto the porous structure of the plating agent. The plating agent can have high affinity towards oil or wax based, high viscosity products such as honey, plant oil, and the like. The disclosed emulsion can also be plated onto this agent, thus creating a water dissolvable powder.

For larger scale production, a machine capable of high shearing, spraying/misting emulsion in, blending, grinding, heating, and outputting can be used. A fluid drying bed, such as from Freund Vector can be used to produce this powder at large scale with higher consistency. The machine can have a large internal volume where heat can be applied. The powder substrate, for example, N-Zorbit™ 2144, can be placed inside the internal volume. There is a high-speed air incoming from either the top or bottom of the volume, while the emulsion is sprayed from the opposite of into the high-speed air, which stirs up the powdery material to well mix the emulsion onto the powder evenly. When high temperature is applied inside the machine, such as 40-80° C., the water content from the emulsion can dry quickly thus allow the powder to be dried while the plating mechanism completes. This process combines the plating and drying at the same time, in the same place, which can make the process very efficient and scalable.

The emulsion used in this application can have the following ingredients:

TABLE 9
Emulsion for powder products

| Ingredients | Mass (g) |
|---|---|
| Active Agent | 1 |
| Carrier oil | 1.5-3 |
| Quillaja extract | 0.075-1 |
| Preservative/Stabilizer | 0-0.1 |
| Water | 2-10 |

Some embodiments of the invention relate to a method of making a powder product:
Step 1. Provide an emulsion with one or more active agents
Step 2. Provide a plating agent
Step 3. Mix the plating agent with the emulsion

Quillaja emulsion can be infused onto the N-Zorbit™ 2144 from 10% up to 75%. For certain application where powder needs to be dry and flowy, the emulsion amount should be below 30%, but for application such as baking or gummy infusion, the emulsion loading percentage can be between 10%-75%.

Further information can be found in PCT Application No. PCT/US2019/041965, filed Jul. 16, 2019 and U.S. application Ser. No. 16/206,869, filed Nov. 30, 2018, which are both fully incorporated by reference herein.

### EXAMPLES

### Example 1

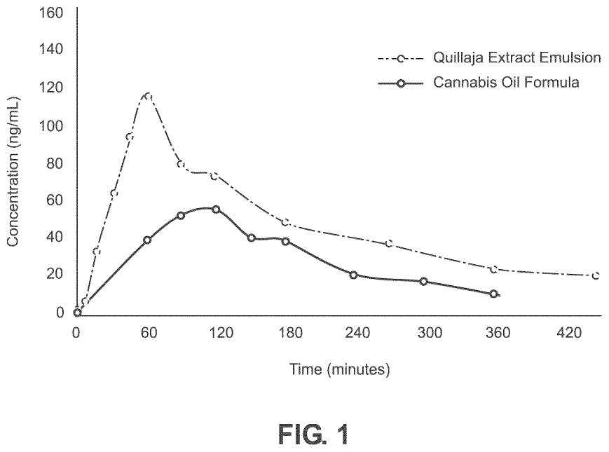

Experiments were done to compare the bioavailability of the inventive emulsion with a standard Cannabis oil formula. 12 subjects were given the CBD-based Quillaja extract emulsion on one day and a CBD isolate dissolved in MCT oil on another day. At each testing day, the subjects were tested under “fed conditions.” After consuming the emulsion, 5 mL of blood was drawn from each tester at 5 minutes, 10 minutes, 15 minutes, 30 minutes, 1 hour, 1.5 hour, 2 hours, 2.5 hours, 3 hours, 4 hours, 5 hours, 6 hours, and 7 hours. The plasma was then separated from the blood by centrifuge and the cannabinoid was extracted by organic solvent before being tested on HPLC-MS-MS for potency. FIG. 1 shows pharmacokinetic study results where it was demonstrated that the Quillaja extract based emulsion has 4-8 times higher total bioavailability than pure MCT oil formula, and the T-max of the Quillaja extract emulsion is around 60 minutes whereas the T-max of MCT formula is around 120 minutes. This result proves the Cannabis emulsion produced from Quillaja extract delivers a much higher bioavailability.

### Example 2

### Target Potency

A starting potency of the Cannabis emulsion at 20 mg/g was used to produce a 10 mg/12 oz beverage. The target potency of the beverage was 10/355=0.028 mg/g. From 20 mg/g to 0.028 mg/g would need 714 times of dilution. In other words, for every 12 oz (355 mL) beverage, the volume of the emulsion is 0.5 mL, which is only 0.14%. This offers enough leftover volume (354.5 mL or 99.86%) for the beverage maker to add flavor, other functional ingredients, preservatives, and water.

The same 20 mg/g emulsion is used to produce a cosmetic product at 300 mg/2 oz. The target potency of the cosmetic product is 5 mg/g (300 mg/60 g). Thus, the dilution factor will be 4 times (20/5). In other words, for every 2 oz (60 g) unit, there will be 15 g or 25% (300 mg/20 mg/g) as emulsion and the rest 45 g or 75% as other topical ingredients. Depending on the topical formulation, it may or may not leave enough room for other topical ingredients.

### Example 3

A Cannabis mouth spray is produced. The mouth spray has a low total volume and the small dimension of the packaging allow the product to fit into a typical clothing pocket, thus possible for discreet and on-the-go use. The spray has a total volume of 5 g. There are 50 sprays per container and each spray has a target potency of 4 mg. The total cannabinoids is 50×4=200 mg. Thus, the overall potency of this product is 200 mg/5 g=40 mg/g. In this case, an emulsion with a starting potency of 20 mg/g will not work. Considering most Cannabis emulsions are bitter by themselves, to formulate an appealing product, enough volume should be left for flavor and stabilizing agents. Therefore, the real volume of the emulsion may only be 2 g out of the 5 g. In such an embodiment, the starting emulsion potency has to be equal to or greater than 100 mg/g (200 mg/2 g).

The mouth spray is produced with different ratios of active ingredients (e.g., cannabinoids, terpenes, and other functional ingredients), which facilitate different targeted feelings such as energetic, uplifting, calm, balanced, focus, and/or the like, and any combinations thereof. Those feelings then can be coupled with real-life experiences such as hiking, attending concerts, exercise, gaming, watching movies, taking long distance flights, and so on. Each mouth spray was designed and colored to indicate different types of feelings and scenarios for which they can be used in.

### Example 4

This example provides guidelines for producing a Cannabis powder using N-Zorbit® 2144 as the plating agent. N-Zorbit® 2144 is a plating agent that mostly consists of modified food starch. It has a very porous structure, wherein billions of small pores can physically absorb the liquid agent that is plated on it.

N-Zorbit® 2144 is usually used to produce a powder from an oil-based input, such as honey, agave, caramel, or other flavor concentrates. The plating procedure is simply infusing and mixing under shear force. A blending equipment as small as KitchenAid® stand mixer, a spray dryer, a heating dryer, or a fluid bed system can all be used in the process where the active input, such as honey, is added into the N-Zorbit® 2144 under constant shear mixing. There is a top oil load percent that N-Zorbit® 2144 can take on without the “wetting”, which varies between different input materials. For honey, the maximum loading percent is around 30-40%, while other flavor systems such as essential oils can be up to 50%. Table 10 below summarizes the maximum load % of different inputs when creating a N-Zorbit® based powder.

TABLE 10
Max active load onto N-Zorbit ® 2144

| | | Maximum |
|---|---|---|
| | | Load % onto |
| Active types | Active Name | N-Zorbit ® 2144 |
| Oil-Based | Honey | 30-40 |
| Input | Agave | 25-35 |
| | Caramel | 25-40 |
| | Essential oils | 40-50 |
| | MCT oil | 40-50 |
| | Cannabinoids in MCT | 40-50 |
| Cannabis | Vitamin E TPGS Emulsion | 10-15 |
| Emulsion | Polysorbate Emulsion | 10-15 |
| Based Input | Polyglycerol Emulsion | 20-25 |
| | Span Emulsion | 20-25 |
| | Gum Acacia Emulsion | 20-25 |
| | Quillaja Extract Emulsion | 30-35 |

There are two ways to create a Cannabis powder using N-Zorbit® 2144: oil formula infused and water formula infused.

### Oil Formula Infused:

Cannabinoid can be dissolved into a carrier oil such as MCT or LCT oil. It is recommended to add additional antioxidants into the oil phase due to the oxidation exposure of the oil on the N-Zorbit® 2144 powder. Not all oil soluble antioxidants work well. The inventor discovered that when combining alpha tocopherol and rosemary extract together, the antioxidant property can reach a maximum, whereas 3-month potency only drops 5% when the powder is stored under sunlight at room temperature without oxygen protection. An oil formula infused powder is easy to produce and involves simple tools like shear mixing. However, it is not a water-soluble powder, since when it is reintroduced into water, the N-Zorbit® 2144 will dissolve but the oil part will float to the top. So, this powder is not used for instant beverage infusion mix, but can be used for tableting, gel capsules, and similar dry applications.

### Emulsion Formula Infused:

To make a truly water-dissolvable powder, the introduced active can be in a water dissolvable form. When an emulsion is infused into the N-Zorbit® 2144 powder, this can be achieved.

Infusing the emulsion into N-Zorbit® 2144 can take the following 5 steps for a lab scale production:
Step 1: Produce the emulsion.

The right type of emulsion determines the overall properties of the final powder.

The potency of the final powder is determined by amount of emulsion applied×emulsion potency/(total weight of the loaded powder). As the finished powder needs to be infused as an ingredient into other products (e.g., stick pack, powdered tea, or coffee) the powder needs to have a starting potency high enough. In order for the finished powder to have suitable potency, the starting potency of the emulsion needs to be as high as possible. Table 11 below shows the range of each ingredient where a 112 mg/g to 279 mg/g CBD emulsion can be produced.

TABLE 11

| CBD | | Quillaja | | | CBD |
|---|---|---|---|---|---|
| isolate | MCT | Extract | Water | Total Weight | potency |
| (g) | (g) | (g) | (g) | (g) | (mg/g) |
| 1 | 0.3-1 | 0.075-0.3 | 2-5 | 3.575-8.8752 | 112-279 |

Step 2: The emulsion is added into the N-Zorbit® 2144 under constant shear force mixing.

It is recommended to add the emulsion by a mist spray instead of pouring directly. The spray ensures the small water droplets do not cause extra clumps during production. At this phase, even when applying mist spray, there will be clumps or chunks from production, which is usually caused by water part of the emulsion hitting the N-Zorbit® 2144 powder.
Step 3: Sieve out the chunks and separate them from the fine powder.

Leave the chunks at 40° C. for 2-12 hours. This step removes moisture from the chunks without decreasing the potency of the cannabinoid(s).
Step 4: Grind the chunks into fine powder using a double-bladed grinder.
Step 5: Reintroduce the grinded chunks to the fine powder and mix them up to average the potency.

Different amounts of Q-Naturale® Quillaja extract based emulsion were added into the N-Zorbit® 2144 powder base and the threshold of emulsion amount was determined by visual inspection of the wetness of the powder and flavor profile.

TABLE 12

| Emulsion | | | | | |
|---|---|---|---|---|---|
| Plated | Emulsion | Powder | Target | Tested | Off from |
| to N-Z ratio | Weight | Weight | Potency | Potency | Expected |
| 10% | 7.5 | 67.5 | 16.6 | 14.39 | −13.31% |
| 15% | 11.25 | 63.75 | 24.9 | 26.35 | 5.82% |
| 20% | 15 | 60 | 33.2 | 40.85 | 23.04% |
| 25% | 18.75 | 56.25 | 41.5 | 46.77 | 12.70% |
| 30% | 22.5 | 52.5 | 49.8 | 56.33 | 13.11% |
| 35% | 26.25 | 48.75 | 58.1 | 66.14 | 13.84% |
| 40% | 30 | 45 | 66.4 | 77.65 | 16.94% |
| 50% | 37.5 | 37.5 | 74.7 | 75 | 0.4% |
| 60% | 45 | 30 | 83 | 86 | 3.6% |
| 75% | 56.25 | 18.75 | 93 | 99 | 6.4% |

Observation from the samples in the table showed that 35% and 40% powders lead to some N-Zorbit® 2144 coated to the side of the mixing bowl. A more refined process for scraping off this portion will be key in an expanded production run. Potency was consistent at each level, indicating all powders were processed in a manner that did not lose active emulsion. Powders were sieved at the micron level of 2000, 500, 250, 125, and 63. All powder chunks >500 micron were set aside and ground using a blade coffee grinder. These were then reincorporated and mixed with the rest of the powder for potency testing.

Visually, the 35% and 40% emulsion plated to N-Zorbit® 2144 ratio samples were a more yellow or off-white color due to the emulsion load. The 10% and 15% samples seemed to have plating agent that was not being utilized. In terms of the water solubility test, all samples were made at ˜20 mg per 12 oz in both hot and room temperature water. Lower load levels performed more poorly in hot water, likely due to excess of plating agent present. Large chunks formed in the water and did not dissolve, most notably in the 15% hot water. High load levels dissolved quickly in hot water. All samples had some particulate matter after stirring. Differences in hot water were more apparent than room temperature.

The color of the powder changed from light yellow to dark yellow when the emulsion level increased from 10% to 40%.

Table 13 shows that different emulsion systems bind with N-Zorbit® 2144 differently. Based on the maximum potency from each emulsion type, the estimated maximum potency of the powder was calculated. The Quillaja extract emulsion produced a powder with potency higher than 50 mg/g.

For a 5 g final product with a 20 mg cannabinoid target, 2.5 g can be sugar, 1 g can be electrolytes, and 0.5 g can be other stabilizers. This would leave 1 g for Cannabis powder. In this case, the Cannabis powder, such as the previously described powder made from Quillaja extract, needs to be at least 20 mg/g to fulfill this formulation.

TABLE 13
Different emulsions' compatibility & potential
target potency with N-Zorbit ® 2144

| | | Maximum | Maximum |
|---|---|---|---|
| | Maximum | Potency | Potency |
| | Load % onto | of Emulsions | of Powder |
| Active Name | N-Zorbit ® 2144 | (mg/g) | (mg/g) |
| Vitamin E TPGS | 15 | 25 | 3.3 |
| Emulsion | | | |
| Polysorbate | 15 | 30 | 3.9 |
| Emulsion | | | |
| Polyglycerol | 25 | 20 | 4.0 |
| Emulsion | | | |
| Span Emulsion | 25 | 30 | 6.0 |
| Gum Acacia | 25 | 40 | 8.0 |
| Emulsion | | | |
| Quillaja Extract | 35 | 279 | 72.4 |
| Emulsion | | | |

Below is a description for using a fluid drying bed to produce a water-soluble Cannabis powder using high potency Quillaja extract emulsion and a porous base substrate (e.g., modified food starch, maltodextrin, and the like). Surprisingly, the ideal emulsion load onto the substrate does not follow a simple, predictable trend.

In an experiment in which Quillaja extract emulsion was sprayed onto modified food starch, the run volume was 8 liters of dry powder. The bulk density was 0.42 grams per liter, so 3.374 kilograms N-Zorbit® (modified food starch) was used. N-Zorbit® was loaded into VSC-3 Lab drier and sealed, with the Quillaja extract emulsion loaded onto the integrated scale to measure total weight sprayed. The total emulsion weight added was 3.496 kilograms. The goal was to determine optimal load level of active onto plating agent, with a maximum target of 22% oil load by dry weight. Samples were pulled periodically as load level increased to test flowability, moisture content, and appearance. Sample load levels were 5% oil load, 10% oil load, 15% oil load, and 20% oil load as final powder. As oil load increased, flowability decreased and powder started to look and feel like kinetic sand. Run was stopped at 20% load level, as powder was deemed fully saturated.

In another experiment, Quillaja extract based emulsion was sprayed onto maltodextrin as powder base. Upon comparison of the load level samples, 12% oil load level was identified as the optimal load level for creating high potency powder with strong flow characteristics. Total maltodextrin weight was only 1.711 kilograms. The bulk density was 0.383 grams per liter, so approximately 4.5 liters of Maltodextrin was loaded into the product chamber. Total emulsion weight added was 1.064 kg. Maltodextrin has been widely used as a substrate when producing a water-soluble powder. Surprisingly, maltodextrin had much higher static cling than modified food starch (N-Zorbit®) when it comes to working with Quillaja extract based Cannabis emulsion, making it stick to the side walls of the expansion chamber. Furthermore, in comparison to the N-Zorbit®, the maltodextrin seemed to get oily and thicker faster. Samples were pulled at 4% and 8% load levels to monitor the characteristics of the powder. At the 12% load level, physical characteristics were more similar to the 20% N-Zorbit® powder from day 1. Run was stopped at 12% and particle size was tested. Final run was completed with N-Zorbit®, with a load level of 12% as the final goal. The bulk density was 0.42 grams per liter, so 3.374 kilograms N-Zorbit® was used. N-Zorbit® was loaded into VSC-3 Lab drier and sealed, with the emulsion loaded onto the integrated scale to measure total weight sprayed. Total Quillaja extract based emulsion weight added was 2.023 kilograms. Run was much more efficient than day 1, with less emulsion to load and a baseline for parameters both contributing to quicker processing time. Total run time was 111.15 minutes, with the parameters used in trial 3. Samples were pulled at 4% and 8% load level and saw similar trends to day 1 samples, thus allowing us to continue to 12% load level.

Particle sizes are measured and shown with their X10, X50, and X90, which is the diameter of particles that 10%, 50%, and 90% of the powder falls under, respectively. Analysis was carried out using a Sympatec® QICPIC Particle size analyzer.

TABLE 14

| Trial | X10[μm] | X50[μm] | X90[μm]: |
|---|---|---|---|
| 1 | 56.42 ± 1.78 | 110.29 ± 6.13 | 186.87 ± 11.59 |
| 2 | 75.52 ± 2.91 | 130.35 ± 3.91 | 221.04 ± 15.63 |
| 3 | 34.64 ± 4.22 | 67.93 ± 4.60 | 109.12 6.10 |

Moisture content is consistent as the VSC-Lab3 dries the powder while spraying. This shows the efficacy of the technology in creating finished powder. With similar load levels, moisture content should not have much variation.

TABLE 15

| Trial | Product Moisture Percentage |
|---|---|
| 1 | 3.08 |
| 2 | 3.66 |
| 3 | 3.17 |

Bulk and tap density were measured using a 100 mL graduated cylinder and funnel. Bulk Density was measured using WHO's method outlined here. Tap Density was measured approximately using method recommended by Freund-Vector, as a tapped density testing machine was not available.

TABLE 16

| Parameter | Measurement (g/mL) |
|---|---|
| Bulk Density | 0.3522 |
| Tap Density | 0.4877 |
| Compressibility Index | 27.7 |
| Hausner Ratio | 1.38 |

12% Load level N-Zorbit® powder (Trial 3) was sent for full panel testing at a third-party lab to confirm potency and other specifications. A total of 2.023 kilograms of emulsion was added at 200.11 mg/g, meaning the 4.009 kilograms of finished powder should be ˜100 milligrams per gram. The tested potency is lower, indicating there may be emulsion sticking to the sides of the container and that additional validation would be required to increase potency yield closer to theoretical.

TABLE 17

| Testing Parameter | Result |
|---|---|
| Potency | 84.91 mg/g |
| Microbiological Screen + Mycotoxins | Passed |
| Residual Solvent | Passed |
| Heavy Metals | Passed |
| Pesticide Screen | Passed |
| Moisture Content | 4.11% |

A fluid drying bed such as VSC-Lab3 can be a straightforward and powerful piece of equipment for loading high potency Quillaja extract based Cannabis emulsion system onto a powder base. The interface was intuitive and programmable, upkeep seemed well within operating procedures, and powders were of a high quality

### Example 5

Co-emulsifier: Some synthetic emulsifiers, such as polysorbate or polyglycerol, need a co-emulsifier to stabilize the emulsion system. The co-emulsifier can lower the overall surface tension and improve the stability of emulsion droplets.

However, Quillaja extract in the concentrated form and/or diluted form was found to be sufficient to sustain the stability of the emulsion. The higher ratio of the Quillaja extract, the smaller the droplet size and higher overall stability of the emulsion. The more stable, smaller droplet size emulsion made by Quillaja extract is used for beverage infusion to produce products with long term stability. In contrast, emulsions produced by Quillaja extract that are less stable and have a larger droplet size can be used in applications where the dilution factor is low (e.g., mouth spray) or dry applications such as absorbing onto a powdery base or gummy.

### Example 6

Water amount vs. viscosity: For certain emulsifiers to work, there must be an ideal water/emulsifier ratio. Below this ratio, the emulsion can become very viscous and difficult to produce on a large scale.

Most of the emulsifiers, including Vitamin E TPGS, Polysorbate, Polyglycerol, and Span, are wax at room temperature and liquid at elevated temperatures. Each emulsifier has a different water solubility and Critical Micelle Concentration (CMC).

When dissolved in water at the same amount (e.g., 1 g emulsifier dissolved in 5 g water) the viscosities of the solutions vary. When an emulsion is produced under ultrasonication, microfluidic, or high shear mixing, high viscosity can cause issues such as clogging machinery, uneven production formalities, and undesirable products. It is recommended to keep viscosity below 900 cps.

To produce a stable emulsion using Quillaja extract at different potency levels, ultrasonication and/or high-pressure homogenization can be used. When using ultrasonication, the amplitude can be adjusted to 50-100% and sonication time depends on the total volume of the emulsion and when the target droplet size is reached. The larger the batch size and the smaller target droplet size it desires, the longer sonication time is needed. Dynamic Light Scattering can be used to monitor the whole process during the droplet size reduction. A chilling mechanism can be applied during the method to keep the emulsion liquid temperature below 40-70° C.

When applying a high-pressure homogenizer, different brands can be applied including Microfluidic®, BEEi®, Pressure Bioscience® and Dyhydromatic®. The mechanism between these machines are similar, i.e., high pressure is generated and applied in a confined chamber where the shear force is applied onto the raw emulsion as energy input. Some systems have two pumps and some systems have one pump. The two-pump system may allow for better homogeneity of the droplets. The running condition is determined by the processing pressure (PSI) and the number of passes. The higher the PSI and more processing passes, the smaller the droplet and mono-distribution of the droplet. Usually for a Cannabis emulsion based on Quillaja extract, the pressure is used at 10-45K PSI and the number of passes can be anywhere between 1-10, depending on the target droplet size.

The invention relates to the surprising finding that unlike all the other emulsifiers that require high water ratios in order to achieve a reasonably low viscosity, Quillaja extract does not need as high of a water ratio to maintain a solution with low viscosity of the solution (see Table 18).

Quillaja extract, as the surface-active ingredient, comes from the product called Q-Naturale®, which is a 20% Quillaja extract water solution. So, when applying Q-Naturale® as the emulsifier input, 80% of the Q-Naturale® weight contributes to water content. This could be another reason that Quillaja extract does not require a high amount of water to reach low viscosity. Quillaja extract can also come from other commercial products such as SAPNOV® or Q Ultra NP®, which are also water solutions of Quillaja extract.

TABLE 18
Water/Emulsifier Ratio and Targeted Viscosity

| | Minimum | Emulsion Viscosity |
|---|---|---|
| | Water to | at Minimum Water |
| Active Name | Emulsifier Ratio | Level (cps) |
| Vitamin E TPGS Emulsion | 7 | 760 |
| Polysorbate 60 Emulsion | 6 | 690 |
| Polyglyceryl-10 Dipalmitate | 7 | 890 |
| Emulsion | | |
| Span 60 Emulsion | 7 | 670 |
| Gum Acacia Emulsion | 6 | 800 |
| Quillaja Extract Emulsion | 2 | 300 |

### Example 7

6 different emulsifiers were tested to produce a THC emulsion targeting 50 mg/g active potency. The emulsifiers were: Vitamin E TPGS, polysorbate 60, Polyglyceryl-10 Dipalmitate, gum acacia, Quillaja extract, and Span 60. The ingredient ratio was kept the same to cross compare each emulsifier.

A 1:1 carrier oil to CBD isolate ratio was used in the study. To reach 100 mg/g, per 1 gram of CBD isolate, the total weight of the emulsion should be 10 grams to allow for 8 grams to be split between main emulsifier and water. Based on the minimum water/emulsifier ratio in Table 18, the final weight is shown in Table 19 below.

All emulsions were processed under the same condition at 30,000 PSI in high pressure homogenizer such as Microfluidizer® for 2 passes. Afterward, the emulsion was measured for droplet size and put into a 60° C. oven for 48 hours. The droplet size growth % is considered one critical standard to evaluate emulsion heat stability. The data shows that only emulsion made by Quillaja extract maintained the droplet size growth below 5% and all other emulsion types had droplet size growth from 56% to 360%, indicating that those emulsions are not stable.

TABLE 19

| | | | | | | Droplet |
|---|---|---|---|---|---|---|
| | | | | | | size |
| | | | | | | growth % |
| Main | | | Main | | | heating at |
| Emulsifier | CBD | MCT | Emul- | | | 60° C. for |
| Type | isolate | oil | sifier | Water | Total | 48 hours |
| Vitamin E | 1 | 1 | 1 | 7 | 10 | 56% |
| TPGS | | | | | | |
| Polysorbate 60 | 1 | 1 | 1.14 | 6.84 | 10 | 290% |
| Polyglyceryl-10 | 1 | 1 | 1 | 7 | 10 | 159% |
| Dipalmitate | | | | | | |
| Span 60 | 1 | 1 | 1 | 7 | 10 | 360% |
| gum acacia | 1 | 1 | 1.14 | 6.84 | 10 | 60% |
| Quillaja | 1 | 1 | 0.3 | 5.34 | 10 | 2% |
| extract | | | | | | |

### Example 8

Oil load: Active oil load is another key differentiation between Quillaja extract and other emulsifiers. In a simple MCT oil+emulsifier+water system, when MCT is increased, the emulsion droplet size will increase and when emulsifier is increased, the emulsion droplet will decrease. When the MCT/emulsifier ratio is above 1.5-5 times of emulsifier amount, the emulsion loses its stability.

By conducting an experiment to evaluate each emulsifier with increased MCT oil load, it was shown that Quillaja extract can hold and stabilize an oil load roughly 5 times that of other emulsifiers (see Table 20). This is the key reason that Quillaja extract can produce Cannabis emulsions with a high starting potency.

TABLE 20
Maximum oil load of emulsifiers without losing emulsion stability

| | Maximum MCT Oil that can be |
|---|---|
| | emulsified and stabilized |
| Active Name | by 10 g of emulsifier |
| Vitamin E TPGS Emulsion | 12 |
| Polysorbate Emulsion | 10 |
| Polyglycerol Emulsion | 8 |
| Span Emulsion | 7 |
| Gum Acacia Emulsion | 5 |
| Quillaja Extract Emulsion | 50 |

### Example 9

Emulsion stability: When pushing the oil load of the emulsion, the stability of the emulsion can be affected, especially when there is not enough emulsifier to lower the interfacial tension.

Emulsion stability can be evaluated by three steps:
Step 1: Heating the raw emulsion at 55° C.-60° C. for 48 hours and checking droplet size growth. Droplet size growth <10% is a good passing criteria.
Step 2: Centrifuging the emulsion to accelerate gravity stability and observing layer separation, with no visible layer separation as passing criteria.
Step 3: Placing the diluted emulsion at desired pH at room temperature to observe O-ring formation, with no visible O-ring formation as passing criteria.

In Table 20, the maximum oil load shown creates an emulsion that passes all three aforementioned criteria. When oil load increases further, the emulsion starts to lose stability, thus becoming less desirable.

### Example 10

When pushing the limit of starting potency, emulsion flavor can change. For some emulsifiers, the less carrier oil used, the more bitter the flavor of the emulsion can be. The carrier oil is there to block the bitterness that arises from the cannabinoid(s) and emulsifier.

However, for Quillaja extract emulsion, it was found that the less carrier oil used, the less bitter the flavor.

### Example 11

An emulsion using Polyglycerol emulsifier was tested using different active agents. When the active agent was changed from CBD isolate to CBN isolate, and all other factors were kept the same, the emulsion became more viscous and droplet size became 30-50% larger. A similar trend was observed for Polysorbate and Vitamin E TPGS emulsions. It was unexpectedly discovered that emulsions with Quillaja extract offered a wide robustness towards different active input. See Table 21 for details.

TABLE 21
Different active agents affect emulsion average
droplet size under same processing conditions

| | | CBD | THC | CBN |
|---|---|---|---|---|
| | | Isolate | Distillate | Isolate |
| | Emulsion Type | Average Droplet Size (nm) | | |
| “Processed by | Vitamin E TPGS | 50 | 60 | 110 |
| 30 minutes of | Emulsion | | | |
| high shear | Polysorbate | 50 | 70 | 90 |
| mixing + | Emulsion | | | |
| Microfluidic | Polyglycerol | 120 | 150 | 180 |
| at 30,000 PSI | Emulsion | | | |
| for 2 Pass” | Gum Acacia | 450 | 490 | 580 |
| | Emulsion | | | |
| | Quillaja Extract | 175 | 170 | 180 |
| | Emulsion | | | |

To produce a Cannabis emulsion on a large scale, the chemical design (emulsifier and ratio) needs to be robust against temperature, processing pressure, active types, and carrier oil types. For certain emulsion systems, when certain processing pressures or active types change, their emulsion droplet distribution changes. Studies were done to show that emulsions with Quillaja extract offer a consistent droplet size around 150-350 nm regardless of production conditions. This is another surprising advantage that Quillaja extract offers.

### Example 12

The Quillaja-based Cannabis emulsion has a very wide range of emulsion potency target. It can reach anywhere from 0.5% (5 mg/g) to nearly 38% (380 mg/g). Q-Naturale®, which is a ˜20% Quillaja extract water solution, can be used to illustrate emulsion internal ratios.

When designing the emulsion ratios, the carrier oil is usually at least 0.3 times the cannabinoid oil, the Q-Naturale® is usually at least 0.3 times the total amount of cannabinoid(s) and carrier oil, and the water amount is no less than the total amount of cannabinoid(s) and carrier oil minus the Q-Naturale® amount. Based on Q-Naturale® has 18-20% active Quillaja extract, the Quillaja extract is usually at least 0.05 times the total amount of cannabinoid(s) and carrier oil.

Table 25 summarizes the full ratio of what Q-Naturale® (Quillaja extract) can achieve in a Cannabis emulsion.

Emulsions A, B, and C have a Q-Naturale® ratio that is 0.3 times the total amount of cannabinoid isolate and carrier oil, but the carrier oil ratio increases from 0.3 to 1 times of cannabinoid isolate. At this low Q-Naturale® level, the emulsion droplet size may trend towards 350 nm and the viscosity of the emulsion may reach to the upper range at 500 cps. The ratio is usually not recommended for infusion into a ready-to-drink beverage with long shelf life, but it is recommended that the ratio is used for applications that need high potency but small volume (where an emulsion does not need to be diluted many times) such as in a small mouth spray or infusions into N-Zorbit® 2144 to create powder.

Emulsions C, D, and E keep the carrier oil ratio the same, but increased the Q-Naturale® ratio from 0.3 to 0.5 to 0.7 times the total amount of cannabinoid and carrier oil. This change will cause the emulsion flavor to be more bitter, but it will also reduce the droplet size and boost the overall physical stability, which allows for long-term shelf stability and a ready-to-drink formulation.

Emulsion E to emulsion I share the same ratio of carrier oil and Q-Naturale®. In addition, in each of these emulsions, the water amount is increased, which increases the total emulsion weight and causes the overall potency to decrease from 250 mg/g to 5 mg/g. When water amount is higher, the overall droplet size has a slight reduction, but viscosity drops dramatically.

When the carrier oil ratio increases, in emulsion m to emulsion u, it is still possible to produce high potency emulsions by applying similar ratios of Q-Naturale® and water. The Q-Naturale® offers a versatile system that allows for a higher load of carrier oil, where it is necessary to modulate flavor controls. The carrier oil can also be replaced by flavor oil to offer the right flavor of the emulsion for a certain beverage.

For a regular emulsion, potency of 25 mg/g is a comfortable range, when potency increases beyond that level, the emulsion tends to be very viscous and sticky. This creates challenges for large scale production. This proves that the unique feature that Q-Naturale® (Quillaja extract) not only can produce emulsion with potency higher than 25 mg/g but can reach up to 380 mg/g without compromising the overall properties. The actual Quillaja extract amount is around 18-20% of the Q-Naturale® amount according to the active purity.

TABLE 22
Full range of cannabinoids potency that can be targeted by Quillaja extract or Q-Naturale ®

| | | | | | | | Average | |
|---|---|---|---|---|---|---|---|---|
| | | | | | Total | | Droplet | |
| | Cannabinoid | Carrier | Q- | | Weight | Potency | Size | Viscosity |
| Emulsion | Isolate | Oil | Naturale ® | Water | (g) | (mg/g) | (nm) | (cps) |
| a | 1 | 0.3 | 0.39 | 0.91 | 2.6 | 384.6 | 450 | 500 |
| b | 1 | 0.5 | 0.45 | 1.05 | 3 | 333.3 | 400 | 475 |
| c | 1 | 1 | 0.6 | 1.4 | 4 | 250 | 350 | 450 |
| d | 1 | 1 | 1 | 1 | 4 | 250 | 300 | 420 |
| e | 1 | 1 | 1.4 | 0.6 | 4 | 250 | 270 | 350 |
| f | 1 | 1 | 1.4 | 1.59 | 4.99 | 200.4 | 250 | 300 |
| g | 1 | 1 | 1.4 | 3.25 | 6.65 | 150.4 | 240 | 280 |
| h | 1 | 1 | 1.4 | 6.6 | 10 | 100 | 200 | 250 |
| i | 1 | 1 | 1.4 | 16.6 | 20 | 50 | 180 | 100 |
| j | 1 | 1 | 1.4 | 36.6 | 40 | 25 | 150 | 60 |
| k | 1 | 1 | 1.4 | 96.6 | 100 | 10 | 200 | 20 |
| l | 1 | 1 | 1.4 | 196.6 | 200 | 5 | 200 | 10 |
| m | 1 | 2 | 0.9 | 2.1 | 6 | 166.7 | 350 | 450 |
| n | 1 | 2 | 1.5 | 1.5 | 6 | 166.7 | 300 | 420 |
| o | 1 | 2 | 2.1 | 0.9 | 6 | 166.7 | 270 | 350 |
| p | 1 | 3 | 1.2 | 2.8 | 8 | 125 | 350 | 450 |
| q | 1 | 3 | 2 | 2 | 8 | 125 | 300 | 420 |
| r | 1 | 3 | 2.8 | 1.2 | 8 | 125 | 270 | 350 |
| s | 1 | 4 | 1.5 | 3.5 | 10 | 100 | 350 | 450 |
| t | 1 | 4 | 2.5 | 2.5 | 10 | 100 | 300 | 420 |
| u | 1 | 4 | 3.5 | 1.5 | 10 | 100 | 270 | 350 |

The table below summarizes the range of the ingredient ratio and target potency, where proves the huge range of target potency Quillaja extract can deliver as the main emulsifier. This feature enables the versatility of applying this emulsion into many unique infused products mentioned in this application.

TABLE 23

| | | Q- | | | | Average | |
|---|---|---|---|---|---|---|---|
| | | Naturale ® | | | | Droplet | |
| Cannabinoid | Carrier | (Quillaja | | Total | Potency | Size | Viscosity |
| Isolate | Oil | extract) | Water | Weight | (mg/g) | (nm) | (cps) |
| 1 | 0.3-4 | 0.39-3.5 | 0.91-196 | 2.6-200 | 5-384.6 | 150-450 | 10-500 |
| | | (0.07-0.7) | | | | | |

### Example 13

Three different emulsions were produced and evaluated for skin care purposes. They were designed to fit three different skin care categories: “conventional” (polysorbate 60), “natural” (polyglycerol 10-2-P), and “organic” (Q-Naturale®) ingredient source requirement. The ingredient compositions are outlined in Table 24 below:

TABLE 24

| Skin Care | | Carrier | | Polyglycerol | Sunflower | Q- | Wate |
|---|---|---|---|---|---|---|---|
| Emulsion | CBD | oil | Polysorbate 60 | 10-2-P | Lecithin | Naturale ® | r |
| Conventional | 1 | 1-4 | 1.4-3.5 | 0 | 0.7-1.7 | 0 | 20 |
| Formula | | | | | | | |
| Natural | 1 | 1-7 | 0 | 0.5-6 | 0.25-4 | 0 | 20 |
| Formula | | | | | | | |
| Organic | 1 | 1-4 | 0 | 0 | 0 | 1.4-3.5 | 10 |
| Formula | | | | | | | |

These three emulsions were first evaluated for skin irritation and allergy studies using a patch test at a third-party skin care evaluation lab. The three pure emulsion formulas were tested at 20 mg/mL concentration on the patch on 55 subjects.

### Standards for Inclusion in a Study:

Individuals who were not currently under a doctor's care.
Individuals who were free of any dermatological or systemic disorder that would interfere with the results, at the discretion of the Investigator.
Individuals who were free of any acute or chronic disease that would interfere with or increase the risk of study participation.
Individuals who completed a preliminary medical history form and were proven to be in general good health.
Individuals who read, understood, and signed an informed consent document relating to the specific type of study.
Individuals who were able to cooperate with the Investigator and research staff, willing to have test materials applied according to the protocols and completed the full course of the study.
   Standards for Exclusion from a Study:
Individuals who were under 18 years of age.
Individuals who were currently under a doctor's care.
Individuals who were currently taking any medication (topical or systemic) that might mask or interfere with the test results.
Individuals who had a history of any acute or chronic disease that might interfere with or increase the risk associated with study participation.
Individuals who were diagnosed with chronic skin allergies.
Female volunteers.

Below is the information on the subjects:
Number of subjects enrolled: 55
Number of subjects completing study: 52
Age Range: 18-63 years
Sex: Male 11, Female 41
Fitzpatrick Skin Type*
   1—always burn, does not tan 0
   2—burn easily, tan slightly 0
   3—burn moderately, tan progressively 52
   4—burn a little, always tan 0
   5—rarely burn, tan intensely 0
   6—never burn, tan very intensely 0

Panel recruitment selection was accomplished using advertisements in local periodicals, community bulletin boards, phone solicitation, electronic media, and combinations thereof.

The patches were prepared with each of the 3 different emulsion types separately. Each emulsion was prepared at 20 mg/g. The test was focused on the effects of the emulsion without the influence of any other skin care ingredients. The goal was to stress test if pure emulsion can cause any skin irritation. If the results show no irritation, it would be very unlikely for emulsion infused cosmetic product to cause irritation.

Materials to be tested under occlusive conditions were placed on an 8-millimeter aluminum Finn Chamberâ (Epitest Ltd. Oy, Tuusula, Finland) supported on Scanporâ Tape (Norgesplaster A/S, Kristiansand, Norway) or an 8-millimeter filter paper coated aluminum Finn Chamberâ AQUA supported on a thin flexible transparent polyurethane rectangular film coated on one side with a medical grade acrylic adhesive, consistent with adhesive used in state-of-the-art hypoallergenic surgical tapes or a 7 mm IQ-ULTRAâ closed cell system, which is made of additive-free polyethylene plastic foam with a filter paper incorporated (it is supplied in units of 10 chambers on a hypoallergenic non-woven adhesive tape; the width of the tape is 52 mm and the length is 118 mm) or other equivalents.

Test materials tested under semi-occlusive conditions were placed on a test strip with a Rayon/Polypropylene pad or on a 7.5 mm filter paper disc affixed to a strip of hypoallergenic tape (Johnson & Johnson 1 inch First Aid Cloth Tape). Test materials to be tested in an open patch were applied and rubbed directly onto the back of the subject. Approximately 0.02-0.05 mL (in case of liquids) and/or 0.02-0.05 gm (in case of solids) of the test material was used for the study. Liquid test material was dispensed on a 7.5 mm paper disk, which fit in the Finn Chamber.

Below is the procedure of the patch test:
Step 1: Subjects were requested to bathe or wash as usual before arrival at the facility.
Step 2: Patches containing the test material were then affixed directly to the skin of the intrascapular regions of the back, to the right or left of the midline, and subjects were dismissed with instructions not to wet or expose the test area to direct sunlight.
Step 3: Patches remained in place for 48 hours after the first application. Subjects were instructed not to remove the patches prior to their 48-hour scheduled visit. Thereafter, subjects were instructed to remove patches 24 hours after application for the remainder of the study.
Step 4: This procedure was repeated until a series of nine (9) consecutive, 24-hour exposures had been made three (3) times a week for three (3) consecutive weeks.
Step 5: Prior to each reapplication, the test sites evaluated by trained laboratory personnel.
Step 6: Following a 10-14 day rest period, a retest/challenge dose was applied once to a previously unexposed test site. Test sites were evaluated by trained laboratory personnel 48 and 96 hours after application.
Step 7: In the event of an adverse reaction, the area of erythema and edema were measured. Edema is estimated by the evaluation of the skin with respect to the contour of the unaffected normal skin.
Step 8: Subjects were instructed to report any delayed reactions that might occur after the final reading.

Scoring scale and definition of symbols shown below are based on the scoring scheme according to the International Contact Dermatitis Research Group scoring scale listed below:
0 no reaction (negative)
1 erythema throughout at least ¾ of patch area
2 erythema and induration throughout at least ¾ of patch area
3 erythema, induration, and vesicles
4 erythema, induration, and bullae
D Site discontinued
Dc Subject discontinued voluntarily
DcI Subject discontinued per Investigator

Clinical evaluations were performed by a BCS investigator or designee trained in the clinical evaluation of the skin. Whenever feasible, the same individual did the scoring of all the subjects throughout the study and will be blinded to the treatment assignments and any previous scores.

No adverse reactions of any kind were reported during the course of this study. There were eight (8) subjects with a Grade 1 reaction to the positive control (2.0% Sodium Lauryl Sulfate Solution). No subjects showed any signs of reaction to the negative control (DI Water).

Under the conditions of the study, there was no indication of a potential to elicit dermal irritation or sensitization (contact allergy) noted for conventional, natural, and organic skin care formulas.

After confirming that the skin care emulsions did not introduce detrimental effects to users such as irritation and allergy, the next step was to confirm the cannabinoids penetration efficiency of the three emulsions and compare them to an MCT oil formula control. The skin penetration study was also done in a third-party skin research lab using a Franz diffusion cell. Only the pure emulsion was tested in this experiment.

Cadaver skin was used in this experiment, where it was kept by the cell clamp. The emulsion was applied on top of the cadaver skin and given 48 hours to penetrate the skin. Then the sample at the other side of the diffusion cell was collected and analyzed by HPLC for CBD concentration. The cadaver skin was also tape-sampled to distinguish different skin layers and obtain accurate CBD concentration on each layer by HPLC. All three skin care emulsions with the MCT-CBD oil control were evaluated using this procedure.

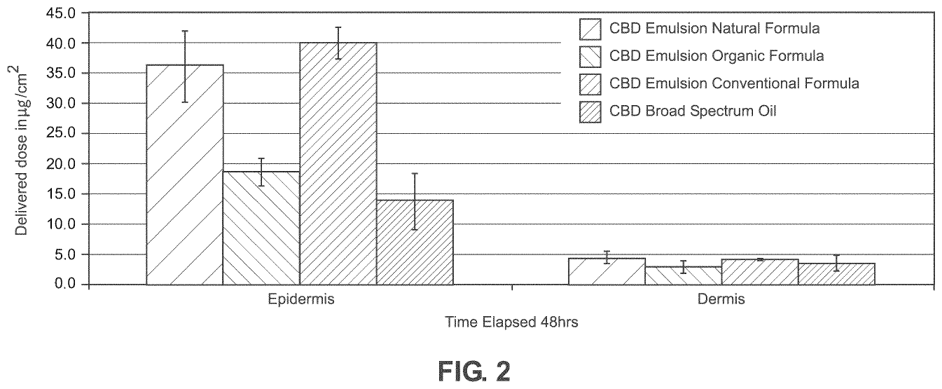

FIG. 2 shows how much CBD was delivered into the epidermis and dermis layer at end of the 48 hours for all formulas. The conventional and natural formula delivered 2.6-3.4 times more CBD than the MCT-CBD control sample. The organic formula delivered ˜30% more CBD to epidermal layer than the control sample. All three emulsions delivered similar amounts of cannabinoids into the dermis layer compared to MCT sample.

The results show that all three emulsion formulas performed better than MCT oil formula in terms of CBD delivery to the epidermis layer, where most skin problems happen (e.g., inflammation, redness, and acne). This is the key to a successful skin care product with cannabinoids.

Also, all three emulsions delivered similar amount of CBD to dermis layer and the amount is very low. This proves that all those formulas do not have the efficacy to be reviewed as the drug, where active can be introduced into the systemic circulation. This reduces the risk of causing a psychoactive effect when applying the emulsion and focusing their end use on skin care.

### Example 14

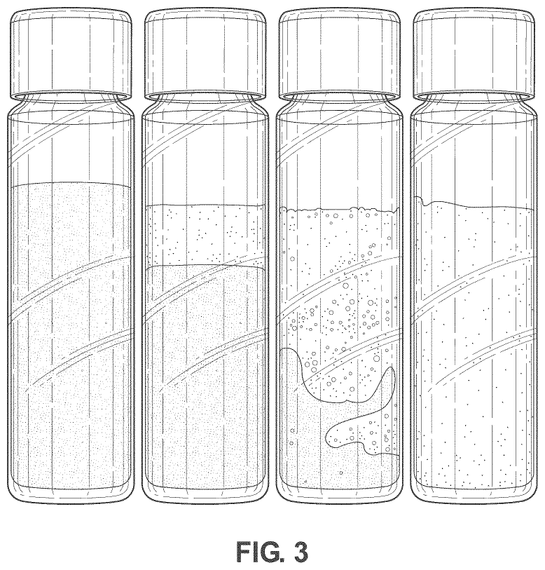

FIG. 3 showcases an example of compatibility observation when different types of emulsions were added into a cosmetic product base. The sample on the left is the product base control, which is a water based facial gel. The three samples on the right contain conventional emulsion, natural emulsion, and organic emulsion. The picture was taken 1 month after infusion and demonstrates that only the organic emulsion (far right) showed no sign of incompatibility.

### Example 15

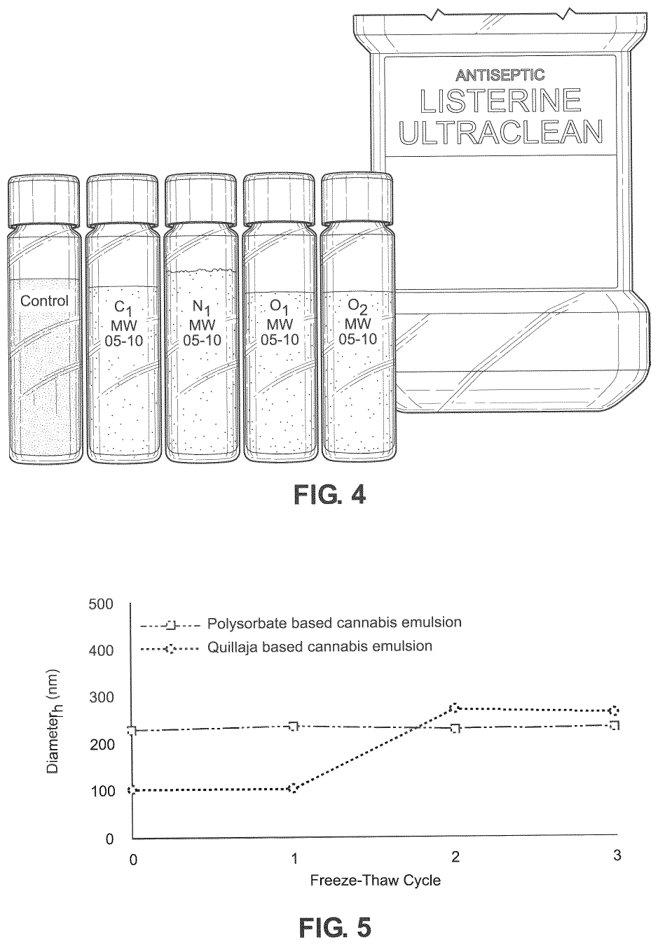

A compatibility study was completed by infusing four types of emulsions with different emulsifiers (Natural 1 (N1): polyglyceryl-10 poleate, Conventional 1 (C1): polysorbate 20 and sunflower lecithin, Organic 1 (01): Quillaja Extract and Organic 2 (02): gum acacia) at 300 mg/1.5 L potency into a mouthwash. The samples were kept at 4° C. for 2 months to monitor physical compatibility. At the end of the study, only N1 showed a slight O-ring in the sample. Conventional 1, Organic 1 and Organic 2 emulsions showed no sign of incompatibility. See FIG. 4.

The emulsion droplet size and the target infusion potency level determine the final turbidity of the product. The turbidity of all the samples were recorded in table below.

TABLE 25

| Turbidity Test | Turbidity (NTU) |
|---|---|
| Control | 4.04 |
| C1 infused | 413 |
| N1 infused | 3322 |
| O1 infused | 1354 |
| O2 infused | 2238 |

### Example 16

Experiments were done to test the compatibility of different co-emulsifiers with Quillaja extract. Co-emulsifiers included Vitamin E TPGS, polyglycerol ester of fatty acid (e.g., Polyglyceryl-10 Dipalmitate, Polyglyceryl-10 oleate, Polyglyceryl-10 laureate, Polyglyceryl-10 caprylate/caprate); sucrose esters (e.g., sucrose laurate, sucrose behenate, sucrose stearate, sucrose palmitate, sucrose erucate, sucrose oleate); lecithin from sunflower, soy, and egg; and polysorbate 20, 60, and 80. Each co-emulsifier was tested at 3:1 ratio to the main emulsifier, which was Quillaja extract. The coarse emulsion was processed under high shear mixer, such as Silverson®, and then processed under high pressure homogenizer at 30,000 PSI for 2 passes.

TABLE 26

| CBD | MCT | Quillaja extract | Co-emulsifier | Water |
|---|---|---|---|---|
| 1 | 2 | 0.33 | 0 or 1 | 5 |

The initial droplet size, droplet size growth after heating 1 day of raw emulsion at 57° C. and emulsion physical compatibility were recorded in the table below. The droplet size growth % should be <10% as the standard as a heat stable emulsion.

TABLE 27

| | | | Visual | |
|---|---|---|---|---|
| | | | observation | |
| | | Droplet size | of Physical | |
| | Initial | growth % | compatibility | |
| Co-emulsifier | Droplet | after 1 day | 2-month post | |
| type | Size (nm) | at 57° C. | emulsification | Conclusion |
| No co-emulsifier | 200 | 2% | No layer | Stable |
| | | | separation | emulsion |
| Vitamin E TPGS | 145 | 2% | No layer | Stable |
| | | | separation | emulsion |
| Polyglyceryl-10 | 150 | 26% | Layer | Unstable |
| Dipalmitate | | | separation | emulsion |
| Polyglyceryl-10 | 161 | 33% | Layer | Unstable |
| oleate | | | separation | emulsion |
| Polyglyceryl-10 | 168 | 30% | Layer | Unstable |
| laureate | | | separation | emulsion |
| Polyglyceryl-10 | 170 | 24% | Layer | Unstable |
| caprylate/caprate | | | separation | emulsion |
| Sucrose laurate | 110 | 8% | No layer | Stable |
| | | | separation | emulsion |
| Sucrose behenate | 165 | 16% | Layer | Unstable |
| | | | separation | emulsion |
| Sucrose stearate | 90 | 6% | No layer | Stable |
| | | | separation | emulsion |
| Sucrose palmitate | 110 | 4% | No layer | Stable |
| | | | separation | emulsion |
| Sucrose erucate | 170 | 19% | Layer | Unstable |
| | | | separation | emulsion |
| Sucrose oleate | 135 | 4% | No layer | Stable |
| | | | separation | emulsion |
| Sunflower | 154 | 8% | No layer | Stable |
| lecithin | | | separation | emulsion |
| Soy lecithin | 123 | 19% | Layer | Unstable |
| | | | separation | emulsion |
| Egg lecithin | 139 | 20% | Layer | Unstable |
| | | | separation | emulsion |
| Polysorbate 20 | 115 | 6% | No layer | Stable |
| | | | separation | emulsion |
| Polysorbate 60 | 105 | 7% | No layer | Stable |
| | | | separation | emulsion |
| Polysorbate 80 | 170 | 40% | Layer | Unstable |
| | | | separation | emulsion |

It was found that only some co-emulsifiers could work in a compatible way with the Quillaja extract system. From the table above, it appears that only Vitamin E TPGS, sucrose laurate, sucrose stearate, sucrose oleate, sucrose palmitate, sunflower lecithin, Polysorbate 20, and Polysorbate 60 can be used as co-emulsifier. With the presence of these co-emulsifiers, the Quillaja extract emulsion droplet size usually has a reduction of 30-110 nm. For example, when Quillaja extract is used along in an emulsion, it can form a stable emulsion with droplet size around 200 nm. And with the presence of co-emulsifier, the initial droplet size can be driven into the range of 90-170 nm. This will increase the transparency of the infused beverage.

### Example 17

Experiments were done to test the use of various essential oils with the emulsion. In these experiments, the essential oil was incorporated with the carrier oil into the oil phase. It was observed that most of the essential oils provided a strong and sharp flavor/aroma when incorporated into a beverage or food product. Orange oil showed the least amount of flavor impact, and since most beverages can easily allow a citric aroma, it works better than other single oils. It was also found that when different essential oils are blended and combined with MCT oil, it caused a less impactful flavor. For example, out of the many combinations, when orange oil is combined with lime oil or key lime oil, at 50:50 to 80:20 ratio, the flavor impact became less. The orange and lime flavors were unexpectedly reduced.

### Example 18

The freeze-thaw stability of various emulsions using different emulsifiers was tested. Surprisingly, only Quillaja extract based emulsion provided desirable freeze-thaw stability with no droplet size change under 3 cycles. Other types of Cannabis emulsion such as polysorbate-based emulsion had significant droplet size growth after 2 freeze-thaw cycles. See FIG. 5.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature, or step, can be employed in various combinations by one of ordinary skill in this art to perform methods in accordance with the principles described herein. Among the various elements, features, and steps some will be specifically included, and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. An emulsion composition for incorporation into a product, wherein the emulsion composition comprises droplets that comprise one or more active agents including at least THC and/or CBD, a carrier oil, an emulsifier, and water; wherein the emulsion composition has a high potency, wherein the high potency is at least 30 mg/g of the active agent or agents, and wherein the emulsifier is a Quillaja extract, and wherein the emulsion is stable in the product.

2. The composition of claim 1, wherein the carrier oil comprises an oil derived from a plant or an animal.

3. The composition of claim 3, wherein the carrier oil is selected from the group consisting of medium chain triglycerides, long chain triglycerides, sunflower oil, olive oil, coconut oil, sesame oil, avocado oil, palm oil, soybean oil, corn oil, peanut oil, canola oil, grape seed oil, hazelnut oil, rice bran oil, linseed oil, safflower oil, passion fruit oil, lard, butter, cheese, animal fat, and combinations thereof.

4. The composition of claim 1, wherein the one or more active agents further comprises a terpene.

5. The composition of claim 1, wherein the one or more active agents comprises a cannabinoid selected from the group consisting of tetrahydrocannabinolic acid a (THCA-A), tetrahydrocannabinolic acid b (THCA-B), tetrahydrocannabinol (THC), tetrahydrocannabinolic acid c (THCA-C), tetrahydrocannbinol c (THC-C), tetrahydrocannabi varinic acid (THCV A), tetrahydrocannabivarin (THCV), tetrahydrocannabiorcolic acid (THCA-C), tetrahydrocannabiorcol (THC-C), delta-7-cis-isotetrahydrocannabivarin, a-tetrahydrocannabinolic acid (A8-THCA), atetrahydrocannabinol (A-THC), cannabidiolic acid (cbda), cannabidiol (CBD), cannabidiol monomethyl ether (CBDM), cannabidiol-c (CBD-C), cannabidivarinic acid (CBDV A), cannabidivarin (CBDV), cannabidiorcol (CBD-C), cannabigerolic acid (CBGA), cannabigerolic acid monomethylether (CBGAM), cannabigerol (CBG), cannabigerol monomethylether (cbgm), cannabigerovarinic acid (cbgva), cannabigerovarin (CBGV), cannabichromenic acid (CBCA), cannabichromene (cbc), cannabichromevarinic acid (CB CVA), cannabichromevarin (CBCV), cannabicyclolic acid (CBLA), cannabicyclol (CBL), cannabicyclovarin (CBLV), cannabielsoic acid a (CBEA-A), cannabielsoic acid b (CBEA-B), cannabielsoin (CBE), cannabinolic acid (CBNA), cannabinol (CBN), cannabinol methylether (CBNM), cannabinol-C (CNB-C), cannabivarin (CBV), cannabino-C (CBN-C), cannabiorcol (CBN-C), cannabinodiol (CBND), cannabinodivarin (CBDV), cannabitriol (CBT), 10-ethoxy-9-hydroxy-a″-tetrahydrocannabinol, 8.9-dihydroxy-a′-tetrahydrocannabinol (8.9-Di-OH CBT-C), cannabitriolvarin (CBTV), ethoxy-cannabitriolvarin (cbtve), dehydrocannabifuran (DCBF), cannbifuran (CBF), cannabichromanon (CBCN), cannabicitran (CBT), 10-oxoa′-tetrahydrocannabinol (OTHC), a-cis-tetrahydrocannabinol (cis-THC), cannabiripsol (CBR), 3,4,5,6-tetrahydro-7-hydroxy-alpha-alpha-2-trim ethyl-9-n-propyl-2,6-methano-2h-1-benzoxocin-5-methanol (OH-iso-HHCV), trihydroxy-delta-9-tetrahydrocannabinol (triCH-THC), isocannabinoids, and combinations thereof.

6. The composition of claim 1, where in the emulsion composition has a high potency, wherein the high potency is at least 40 mg active agent per gram of product.

7. The composition of claim 1, wherein the product is an oral spray, a candy, a small volume beverage shot, or a powder.

8. The composition of claim 1, wherein the product is a cosmetic product.

9. The composition of claim 1, wherein the composition and/or the product is organically certified according to current USDA guidelines.

10. The composition of claim 1, wherein the product has an onset time of less than 30 minutes.

11. The composition of claim 1, wherein the product is a beverage of less than 15 ml with a potency of at least 1 mg active agent per ml of product.

12. The composition of claim 1, wherein the product is a candy comprising a gummy shell and a liquid center, wherein the candy has potency of at least 1 mg active agent per g of product.

13. The composition of claim 1, wherein the product is a powder suitable for consumption.
